(19) United States
(12) Patent Application Publication
(10) Pub. No.: US 2016/0022595 A1
(43) Pub. Date: Jan. 28, 2016

(54) BIOADHESIVE AND BIODEGRADABLE AND FORMULATIONS THAT PROVIDE SUSTAINED RELEASE OF ANTIMICROBIALS, BACTERIOPHAGES AND ANTI-INFLAMMATORY MEDICATIONS FOR INACTIVATION OF BIOFILMS AND THE TREATMENT OF RHINOSINUSITIS AND OTHER INFECTIONS

(71) Applicant: Rhinotopic, LLC, Baltimore, MD (US)

(72) Inventors: Alan H. Shikani, Baltimore, MD (US); Abraham Domb, Baltimore, MD (US); Fred Hassan, Baltimore, MD (US); Syed M. Shah, Baltimore, MD (US)

(21) Appl. No.: 14/808,391

(22) Filed: Jul. 24, 2015

Related U.S. Application Data
(60) Provisional application No. 61/999,344, filed on Jul. 24, 2014.
(60) Provisional application No. 62/179,993, filed on May 26, 2015.

(51) Int. Cl. A61K 9/50; A61K 9/00; A61K 33/30; A61K 31/573; A61K 31/7048; A61K 35/76

(52) U.S. Cl. CPC A61K 9/5036; A61K 31/7048; A61K 35/76; A61K 33/30; A61K 31/573; A61K 9/0043

(57) ABSTRACT

Described is a composition comprising one or more active ingredients coated, dispersed, or dissolved with a mucoadhesive polymer. Although subject to multiple uses, the composition, in some embodiments, is usable for treating rhinosinusitis.

### CROSS REFERENCE

The present application claims priority benefit to U.S. Application No. 61/999,344, file Jul. 24, 2014, which application is hereby incorporated herein by reference in its entirety. The present application claims priority benefit to U.S. Application No. 62/179,993, file May 26, 2015, which application is hereby incorporated herein by reference in its entirety.

### FIELD

Described is a composition comprising one or more active ingredients coated, dispersed or dissolved within a mucoadhesive polymer solution or dispersion. Although subject to multiple uses, the composition, in some embodiments, is usable for treating rhinosinusitis.

### BACKGROUND

Chronic rhinosinusitis (CRS) is one of the commonest chronic diseases, affecting 14.2% of the United States population. CRS places a substantial cost burden on the health care system and is responsible for a considerable portion of sick leaves and decreased productivity. It is associated with over 13 million physician visits per year and an estimated aggregated cost of over $6 billion annually. Patients with chronic rhinosinusitis (CRS) demonstrate worse quality-of-life scores than those suffering from chronic obstructive pulmonary disease, congestive heart failure, back pain, or anginal-3. CRS is believed to have a multifactorial etiology which includes fungi, bacterial superantigens, allergy, aspirin sensitivity, exposure to environmental irritants, and lately, bacterial biofilms. Moreover, conditions impairing the mucociliary function, such as primary ciliary dyskinesia and cystic fibrosis have also been implicated. The resulting chronic inflammation of the sinus mucosa leads to defense reactions and alterations, i.e., edema, high mucus secretion, cilia loss, and particularly, polyp formation.

Surgery to remove the diseased mucosa and open the sinus ostia in order to restore the physiological mucociliary clearance, in combination with systemic antibiotics, has been the mainstay of treatment for the past decades. Opening of the sinus ostia may be reached through traditional endoscopic sinus surgery (introduced in the US in 1985) or through balloon sinuplasty (introduced in the US in 2005). The success rate of both sinus procedures is relatively good, even though it is reported high, anywhere between 85 and 90% during the first year.

Upon longer follow up however, CRS symptoms tend to recur. Interestingly, in the majority of failures, the post-operative sinus anatomy demonstrates ostium patency and wide-open ethmoid cavities, abundantly ventilated. Specifically, Kennedy has reported that 15% of patients, who undergo endoscopic surgery, show mild to no clinical improvement, despite the “optimal” surgical outcome. [Ref. 3] Pooling of the literature points to the following statistics: when followed for up to 3 years after endoscopic sinus surgery, between 5-25% of operated CRS patients exhibit persistent symptoms, even despite optimal medical management and widely open sinus cavities.

These difficult-to-treat patients labeled as “Refractory CRS” sometimes demonstrate inflammatory or idiosyncratic features, such as eosinophilia, history of asthma, allergic fungal sinusitis, nasal polyps, and aspirin sensitivity. The common denominator of the above conditions is an intrinsic pro-inflammatory state of the sinus mucosa which predisposes to clinicopathological exacerbations, in the absence of substantial external irritation. In addition to the aberrations of the end-organ, that is, the sinus epithelium, an unusual issue of resistance of ordinary bacteria to potent antimicrobials has emerged.

This finding has been associated with a breakdown of the integrity of the sinus mucosal membrane in diseased sinuses, and disruption of the tight junctions between epithelial cells which, in a healthy mucosa, form a tight epithelial barrier. Disruption of this epithelia barrier would allow microbes to invade the sub-epithelia stroma and trigger a chronic infection, and/or allow antigens to trigger a chronic sub-epithelial inflammatory process.

This notable finding has been associated with the concept of biofilms, which cover the surface epithelium of diseased paranasal cavities, and may contribute to the disruption of the epithelial barrier. The common bacterial species H. influenzae, S. pneumoniae, and S. aureus have been identified in biofilms, and their capacity to produce this organic matrix correlates with the refractoriness of CRS. Microorganisms colonizing the biofilms are much less vulnerable to systemic antibiotics which reach the standard tissue Minimally Inhibitory Concentration (MIC). Both the physical and chemical protection imposed by the organic layer on the microbial colonies, call for higher local concentrations of the soluble antibacterial agents.

The refractory CRS disease is most likely multifactorial, which chronicity relies on constant debris accumulation, unremitting inflammation, and insidious infection. An optimal management should be multifactorial as well, and address all three components concurrently.

### BRIEF DESCRIPTION OF THE DRAWINGS

FIGS. 1A-B show persistent sinusitis despite widely open sinus cavities.

FIG. 2 shows H&E stains demonstrating biofilms (arrow) in the epithelial surface of the mucosal membrane.

FIG. 3 illustrates and embodiment of the pharmaceutically acceptable composition.

FIG. 4 illustrates and embodiment of the pharmaceutically acceptable composition.

FIG. 5 is a scheme of an embodiment for preparing an ANTIFUNGAL AGENT particles coated with chitosan.

FIG. 6 is a scheme of an embodiment for preparing an ANTIFUNGAL AGENT particles coated with octadecene-maleic anhydride.

### EMBODIMENTS

Reference will now be made in detail to embodiments of the invention, examples of which are illustrated in the accompanying drawings. Wherever possible, the same reference numbers will be used throughout the drawings to refer to the same or like parts.

It is to be understood that both the foregoing general description and the following detailed description are exemplary and explanatory only and are not restrictive of the invention, as claimed.

The accompanying drawings, which are incorporated in and constitute a part of this specification, illustrate embodiments and together with the description, serve to explain the principles.

The inventors noticed that there is mounting evidence that bacterial and possibly fungal biofilms play an important role in the etiology and persistence of CRS. [Refs. 4-8] Although these communities have been associated with a number of diseases in humans, only recently this concept has been applied to chronic rhinosinusitis, and novel anti-biofilm therapies should be developed. Biofilms are a complex, organized community of microorganisms that adhere to the surface of the mucosa and are encased in a host- and pathogen-derived extracellular polymeric matrix that enables them to evade mucociliary clearance and resist oral antibiotics. Several mechanisms have been postulated to explain the enhanced resistance of microorganisms in biofilms, including impaired antibiotic penetration, antibiotic inhibition and/or deactivation, decreased microbial metabolic activity and genetically-transferred antibiotic resistance. [Ref 7-8]

If bacterial biofilms are the cause of certain cases of CRS, then the treatment paradigms will have to include therapies that disrupt biofilms and prevent quorum sensing. Topical sinus therapy that targets microbial biofilm formation in CRS has been to be effective in disputing biofilms.

The principle of the topical sinus therapy is prolonged delivery of a highly concentrated medication locally and directly to the sinus cavities, so as to exert its maximal effect on the desired anatomical site, without significant systemic toxicity.

There has been recently an explosion in the understanding of the mechanisms of chronic rhinosinusitis. Multiple approaches to control and modify the inflammatory and infectious reaction in chronic rhinosinusitis have led to multiple antimicrobial and anti-inflammatory agents being introduced topically to the sinonasal cavities. At the present time, a variety of small to medium-sized compounding pharmacies have been providing patients with a customized approach to antimicrobial and anti-inflammatory topical medications to treat the sinuses. Most of these pharmacies have focused on selling to patients culture-driven antimicrobials and corticosteroids liquid solutions that are delivered to the sinus cavities through a nebulizer. The challenges are: 1. Access is a challenge as these nebulized medications do not always reach far enough into the different sinuses to treat the target mucosa; 2. Sustained availability is a challenge, as the medications are often rapidly disposed of by the muco-ciliary clearance mechanism that sweeps the mucous blanket out of the sinuses; 3. There is no assurance of sterility or reduction in potential infective organisms of the solution/dispersion solution used in the nebulizer; and 4. There is potential for degradation of the antimicrobial and/or anti-inflammatory during the extended period between the compounding in the pharmacy and use by the patient.

The inventors noticed, from reviewing topical sinus and rhinosinusitis therapies, the following. A thorough review on topical drug delivery for CRS is presented by Shikani et al. [Ref 9] and Liang et al. [Ref. 10]. In the history of rhinosinusitis, topical therapy, several methods for drug delivery have been utilized: fluid irrigation, spray pumps, drops/powder/gel instillation, nebulization, and regional installation, aim to provide optimal spatiotemporal conditions of contact between the medication and its target. Commercially available nasal sprays (such as the corticosteroids nasal sprays) have been classically used to provide local application of drugs in rhinosinusitis. Among the various devices developed over the years (spray bottles, aqueous pumps, dry powder atomizers), aqueous spray pumps are most accepted. Such pumps contain a medication-containing solution, which is released in the form of droplets. Smaller, lighter droplets demonstrate a broad distribution across the mucosal surface, as they travel a longer distance from the nostril. The viscosity of the solution is an additional factor, as thicker liquids project in a narrower cone and do not reach the peripheral intranasal regions. Despite the refinements of spraying pumps, the droplets barely penetrate the sinuses in unoperated patients, and their effect is essentially restricted to the nasal cavities only. Most of the sprayed agent is detected in the anterior nasal cavity, due to the obstructive mass of the inferior turbinate. A significant portion of the dose unfortunately does not approach the ostiomeatal complex, an anatomical structure that is central to the pathogenesis of CRS. [Refs. 11-18]Sinus Surgery is a prerequisite for effective sinus topical drug delivery, as the delivery of topical solution to the non-operated sinuses is very limited. The frontal and sphenoid sinuses are essentially not accessible prior to surgery. Surgery is an all-important factor for access, because it gives nebulized the agents a big enough opening and allows the irrigating fluids access of to the sinuses. Typically, an opening that is 4 mm or larger is needed for better results. [Ref. 11] Fluid irrigations remain a traditional, simple, and well-tested technique for conveying treatment formulas directly to the sinonasal surface epithelium. Pressurized nasal spray provides only nasal cavity penetration at best, and squeeze bottle and Neti pot irrigation only provide some maxillary sinus and ethmoid sinus penetration. This heterogeneity creates a confounding variable in determining the effectiveness of topical drug delivery in post-surgical sinus cavities. Studies have shown that irrigation with douching or bulb irrigation is a little more effective than sprays, nebulizers, or atomizers in reaching post-operative sinus cavities, but still not quite adequate. In the operated sinuses, where the ostium has been opened, irrigations with a bulb syringe are superior to every other delivery methods, in terms of access to anatomical subsites. Yet, up to 30 mL of solution pour out immediately from the nasal cavities, so that a considerable irrigation volume is wasted. Low-pressure lavage using commercial pots or high-pressure douches delivered by squeeze-bottles are proper in case of surgically created open cavities, although they may have limited reach.

Nebulized medications are another approach to deliver medications to the sinuses and treat rhinosinusitis that is relatively new in the United States; nebulizers and nebulized medications are covered by most medical insurances. Nebulization devices provide an aerosolized mist which is created by a mechanical pulse. The latter is produced either by a high-pressure jet, ultrasonic vibration or a vibrating mesh. The earliest devices emitted an aerosolized stream of particles larger than 10 μm, and the penetration of medications into the sinuses was limited as most of the particles are filtered by macro- or micro-anatomical barriers Innovative technologies are now capable of generating airflow consisting of particles with a diameter less than 3-4 μm, and accumulation on sinus mucosa is much more significant, however the smaller particle size results in a more significant pulmonary inhalation and the potential for higher systemic drug absorption. The main advantage of nebulizers, in comparison with the traditional spray pumps, is a better deposition of pharmacological agents in the posterior nasal cavity, however the delivery and bioavailability of drugs to the target mucosa is still not quite adequate. Drug access to the sinus mucosa could be a challenge: sprayed formulations are practically undetected in the sinus cavities of patients who have not had surgery, whereas 8% of intranasally placed aerosols remain in the sinuses. The main factors associated with particle penetration include the size of the sinus ostia, the size of the particle, and the flow rate of the aerosol. [Refs. 19,20] Particles >10 μm in size usually do not pass the nasal cavity, and particles <5 μm in size are more likely to enter the sinuses, but they will also reach the lungs. Hyo et al. theorized that ideal particle size for maxillary sinus penetration is between 3 and 10 μm. [Ref 19]

Typical nasal pump sprays generate droplets of 50-100 μm in diameter size, and deliver 70-150 μl of drug per puff, at standard velocities of 7.5-20 L/min. A large fraction of the spray is deposited in the anterior nasal cavity without any significant penetration into the paranasal sinuses. [Refs. 19,20] Furthermore, half of the aerosol is cleared after approximately 15 min, with minimal activity remaining after 6 h. [Ref 21]. Nebulizers deliver medication in mist form, and are commonly used to delivery drugs to the lower airway. A variety of nebulizers have been developed for targeted sinonasal drug delivery. SinuNeb™ (PARI Respiratory Equipment, Midlothian, Va., USA) is a passive-diffusion system; ViaNase™ (Kurve Technology, Lynnwood, Wash., USA) is a vortex-propelled system [Ref. 17] and PARI Sinus™ Pulsating Aerosol System (PARI, Starnberg, Germany) is a pulsating nebulizer that has refined particle size distribution and flow rate. [Ref. 24] OptiNose™ is a breath-actuated bidirectional delivery device (OptiMist™; OptiNose, Oslo, Norway) [Ref. 22, 23]. Studies on the pulsating aerosol system demonstrated improved posterior nasal cavity deposition with access to the ostiomeatal complex and slower clearance times compared with nasal pump sprays, but still the delivery and bioavailability of drugs to the target sinus mucosa is not quite adequate.

It is believed that the pathogenesis of rhinosinusitis is often a combination of infection and inflammation. It has been suggested that an intact epithelial barrier with tight epithelial junctions is helpful for a healthy nasal mucosa. A defect in this barrier allows antigen passage across the epithelium, which subsequently promotes inflammation. [Ref 25] Infection by mucosal biofilms on the epithelial membrane may cause disruption of the tight junctions that are critical to the integrity of the barrier, and subsequently triggers an underlying inflammatory/infectious cascade; this further damages the epithelial membrane, exacerbates the inflammatory/ infectious cascade and eventually leads to persistent/ refractory rhinosinusitis.

The optimal treatment should hence address both infectious and inflammatory commonest. A sustained, highly concentrated application of corticosteroids directly onto the diseased mucosal membrane treats the inflammation-related changes. The sustained, application of antimicrobial agents aims to eradicate the etiologic microorganism from the sinus mucosa, along with microbial biofilms which contribute to the pathogenesis of refractory CRS. [Refs. 26-28]

A factor for the successful elimination of infection is overcoming the resistance of bacteria within the biofilm shelter by locally delivering sustained high levels of antibiotics a to the target mucosa, so that to reach supra-MIC (minimum inhibitory concentration) levels to inactivate the microorganism inside the biofilms.

Another factor is treating the excessive inflammatory reaction that exacerbates mucosal damage perpetuates the infection. This can be achieved by locally delivering sustained high level of corticosteroids which have powerful anti-inflammatory effect.

. An alternative method to treat the bacterial infection in chronic rhinosinusitis is to deliver locally viral bacteriophages. Phages are naturally occurring viruses that attack bacteria. Phages have the ability to diffuse through the biofilm matrix [Ref 6], facilitating phage access to biofilm-bound cells, which are subsequently infected and destroyed by the phage. Phages have been shown in numerous studies to be effective against biofilms, including biofilms of S. aureus.

The challenge is to be able to deliver the phages in a sustained fashion over a period of time, as they can be quickly flushed out of the sinuses otherwise by the local muco-ciliary clearance mechanism of the sinuses. The challenges in any placing topically active agents are as follows:
1. Access: as these nebulized agents do not always reach far enough into the different sinuses to treat the target mucosa;
2. Sustained bioavailability: as the agents are often rapidly disposed of by the muco-ciliary clearance mechanism that sweeps the mucous blanket out of the sinuses, or by gravity when the patient is in the sitting or standing position;
3. The sinus mucous barrier caused by from mucous coating the sinuses may limit the effectiveness of delivery of the agent locally to the target mucosa; and
4. An additional challenge is to be able, in the case of medications, to deliver of sustained high levels of antibiotics to the target mucosa, so that to reach supra-MIC (minimum inhibitory concentration) levels to and overcome the resistance of microorganism inside the biofilms, along with high sustained high levels of corticosteroids in order to reverse the excessive inflammatory reaction that exacerbates mucosal damage perpetuates the infection.

The inventors suggest that one solution is the direct application drug (and/or phage) via some sort of drug-releasing vehicles (or phage-releasing vehicle), including a drug-carring gel, a biodegradable polymers, a polymer-coated stent, or drug-loaded lipospheres would adhere to the mucosa and result in a sustained drug delivery, at high local concentrations, directly to the target mucosa, than aerosolized or nebulized medications. These releasing vehicles would be applied directly to the target mucosa by the treating physician under endoscopic visualization. This would overcome the limitations of aerosolized or nebulized medications (which consist of limited penetration, limited bioavailability, and rapid clearance from the sinuses).

Biodegradable drug-eluting stents that deliver sustained corticosteroids to treat nasal polyps have shown favorable early evidence and the level of evidence for some these devices is getting stronger. In animal models, drug eluting stents have shown decreased granulation tissue without any epithelial damage, decreased post-operative osteoneogenesis and stromal proliferation, and negligible systemic absorption. [Refs. 29, 31] Most drug-eluting stents have focused on corticosteroids, but antimicrobial-eluting stents have also been described. [Refs. 33,34] Approved by the USFDA in 2011, the Propel sinus implant (Intersect ENT, Palo Alto, Calif., USA) is a newer bioabsorbable implant that resembles a coil that self-expands in the sinus cavity and releases 370 μg of mometasone furoate over 4 weeks, and the biodegrades. [Ref 34] Prospective double-blinded trials on a bioabsorbable drug-eluting stent used after endoscopic sinus surgery in patients with CRS have shown significantly reduced inflammation and prevention of significant adhesion compared to a control stent. [Refs. 33,34]

Drug-loaded nanomaterials that can be deposited onto the sinus mucosa (and potentially get integrated into the cell membrane), thereby delivering drugs intracellularly are currently being evaluated. Major classes of nanomaterials used in drug delivery include liposomes (small bubbles made of a bilayer of lipids), polymers, micelles, dendrimers, and metallic/ceramic nanoparticles. [Ref 35] The outlook on nanotechnology-based drug delivery is optimistic but significant work still needs to be done, including optimizing the drug release pharmacokinetics, and formulating biocompatible and biodegradable nanoparticles. One significant issue to overcome is the sinus mucous barrier. Mucus coating of the mucosa may limit the effectiveness of local drug delivery, and “mucus penetrating nanoparticles” may need to be formulated to both enhance penetration of the nanoscale barrier, and achieve more uniform and longer-lasting drug delivery to mucosa, work on these “mucus penetrating nanoparticles” is in progress. [Ref 36] Another challenge is the mucociliary clearance mechanism of the sinuses, which is powerful enough to rapidly sweep away particles that are applied to the sinus mucosa. The coating is hence only temporary and disappears with 24 hours, due to a combination of gravity, blowing of the nose, mucociliary clearance, which is a very significant hurdle for effective drug bioavailability. What is needed is a novel mucoadhesive compound that delivers coated particles with specific affinity to sinus mucosal surface, so that the particles stick to the surface and deliver their drug load locally. The specific mucoadhesion surface is designed to retain the particles that reach the mucosal surface of the nose and sinuses.

What is needed is a novel mucoadhesive compound that delivers coated particles with specific affinity to sinus mucosal surface, so that the particles stick to the surface and deliver their drug load locally. The specific mucoadhesion surface is designed to retain the particles that reach the mucosal surface of the nose and sinuses.

Alternatively, the polymer used to form the gel can have properties of muco-adhesion. Such polymers include acrylic based polymers, such as methacrylic acid co-polymers, which can bind to the mucous layer due to its unique pH/solubility characteristics and ionic/hydrophobic interactions.

The muco-adhesive gel may also contain other delivery enhancers to help with delivery of anti-inflammatory or antimicrobial active agents, chelating agents, surfactants or mucolytics so that to disrupt biofilms. These agents include gap junction openers (such as EDTA etc.) and p-glycoprotein pump inhibitors (such as polysorbate 80 etc.). The anti-inflammatory may be dissolved or dispersed in the gel matrix along with the delivery enhancers.

In some embodiments, the chelating agents include ethylenediamine-tetraacetic acid (EDTA), Citric Acid Zwitterionic Surfactant (CAZS), gallium nitrate, desferrioxamine, penicillamine, dimercaprol, etc. Preferred surfactants include Polyethylene glycol 400; Sodium lauryl sulfate; sorbitan laurate, sorbitan palmitate, sorbitan stearate (available under the tradename SPAN.R™ 20-40-60 etc.); polysorbates including, but not limited to, polyoxyethylene (20) sorbitan monolaurate, polyoxyethylene (20) sorbitan monopalmitate, polyoxyethylene (20) sorbitan monostearate (available under the tradename TWEEN.R™20-40-60 etc.); and Benzalkonium chloride. Preferred mucolytic agents include Acetylcysteine and Dornase Alpha, etc.

The above mixture of mucoadhesive polymer, anti-inflammatory and delivery enhancers may be sterilized or pasteurized in a single use container such as a vial or in a form/fill/seal container, to reduce the chances of introducing bad organism when applying the gel. The contents of the vial or other suitable container could be used to mix with a solution of the antimicrobial/antibacterial/anti-infective agent or re-constitute the antimicrobial/antibacterial/anti-infective agent which is available as a lyophilized cake or powder. The above combinations, in some embodiments, could then be used for nasal administration.

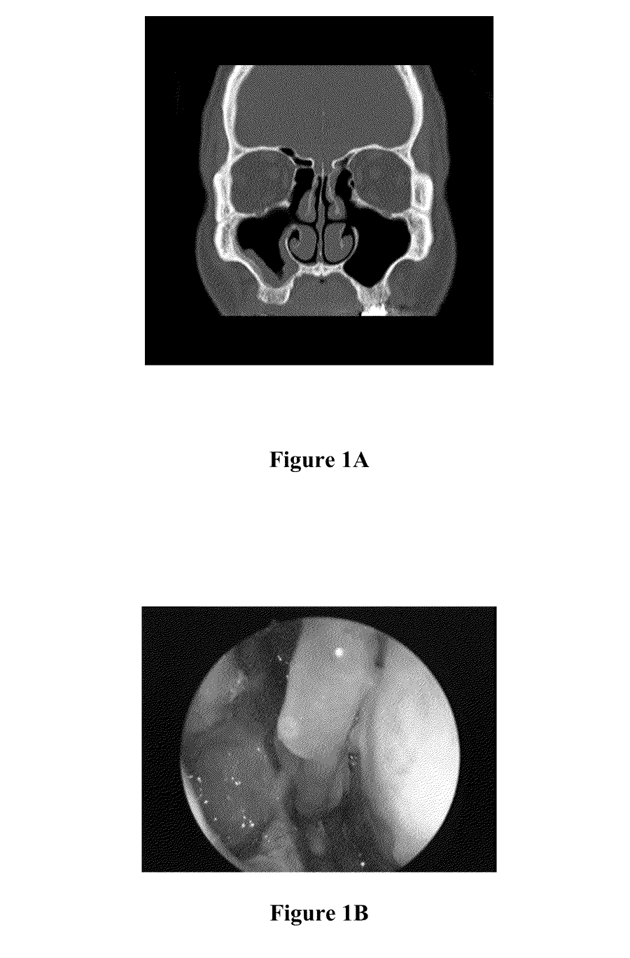

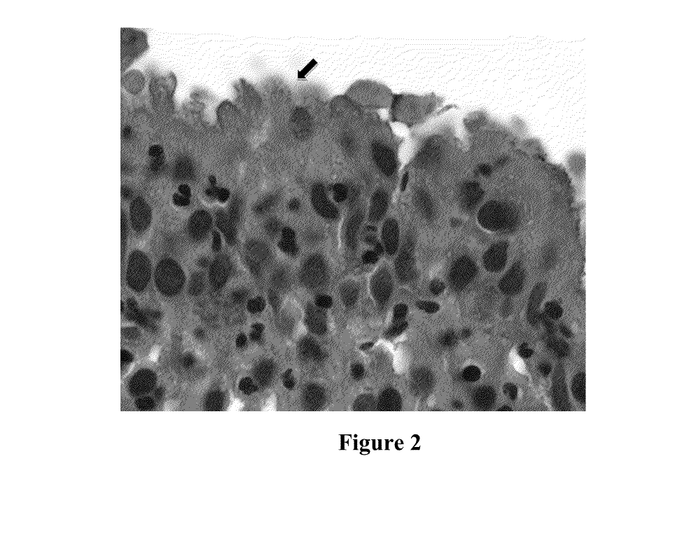

FIGS. 1A-B show persistent sinusitis despite widely open sinus cavities. FIG. 2 shows H&E stains demonstrating biofilms (arrow) in the epithelial surface of the mucosal membrane.

Although discussed in terms of nasal mucosa, in some embodiments, the composition is suitable for administration to other mucosae. For example, in some embodiments, the mucosae is chosen from nasal, oral, gastric, rectal, vaginal, and ocular mucosae.

Described is a pharmaceutically acceptable composition, comprising one or more mucoadhesive polymers coating one or more active ingredients.

In some embodiments, the one or more active ingredients are in the form of solid particles having an average size ranging from 0.050 to 15 microns (μm). In some embodiments, the average size ranges from 0.100 to 10 μm or from 0.900 to 5 μm or from 1 to 3 μm.

In some embodiments, the pharmaceutically acceptable composition is in the form of solid particles having an average size ranging from 0.050 to 15 μm. In some embodiments, the average size ranges from 0.100 to 10 μm or from 0.900 to 5 μm or from 1 to 3 μm.

In some embodiments, the amount of one or more active ingredients ranges from 1 to 50 percent by weight (w/w %) of the one or more active ingredients and one or more pharmaceutically acceptable mucoadhesive polymers. In some embodiments, the amount ranges from 2 to 30 w/w % or from 5 to 20 w/w %.

In some embodiments, the one or more active ingredients are chosen from anti-inflammatory agents, antimicrobial active agents, viral bacteriophages, antihistamines, antiinfectives, and nasal decongestants.

In some embodiments, the anti-inflammatory agents are chosen from steroids and non-steroidal anti-inflammatories (NSAIDS).

In some embodiments, the steroids are chosen from prednisone, dexamethasone, and hydrocortisone.

In some embodiments, the steroids are corticosteroids chosen from prednisolone, prednisone, medrol, beclomethsone, budesonide, flunisolide, fluticasone and triamcinolone. In some embodiments, the anti-inflammatory agents are corticosteroids chosen from dexamethasone, mometasone, and triamcinolone.

In some embodiments, the steroids are corticosteroids chosen from dexamethasone, mometasone, and triamcinolone.

In some embodiments, the NSAIDS are chosen from celecoxib, diclofenac, diflunisal, etodolac, fenoprofen, flurbirofen, ibuprofen, indomethacin, ketroprofen, ketorolac, mefenamic acid, meloxicam, nabumetone, naproxen, oxaprozin, piroxicam, sulindac, and tolmetin,

In some embodiments, the antimicrobial active agents are chosen from antibiotics, antifungals, and anti-virals.

In some embodiments, the antibiotics are chosen from penicillins, cephalosporins, quinolones, aminoglycosides, amphotericin B, etc.)

In some embodiments, the antibiotics such as penicillins, cephalosporins, macrolides, sulfonamides, quinolones, aminoglycosides, betalactam antibiotics, linezolid, vancomycin; aminoglycosides (including amikacin, gentamicin, kanamycin, neomycin, netilmicin, streptomycin, tobramycin, cephalosporins (including cefadroxil, cefazolin, cefalotin, cefalexin, cefaclor, cefoxitin, cefuroxime, cefixime, cefdinir, cefoperazone, cefotaxime, cefpodoxime, ceftazidime, ceftibuten, ceftriaxone, cefepime, loracarbef, ceftaroline ceftobiprole) macrolides (including azithromycin, clarithromycin, erythromycin); penicillins (including amoxicillin, ampicillin, carbenicillin, cloxacillin, dicloxacillin, methicillin, mezlocillin, oxacillin, penicillin, piperacillin, ticarcillin); polypeptides (including bacitracin, colistin, polymyxin b), quinolones ciprofloxacin, levofloxacin, moxifloxacin, norfloxacin, ofloxacin, gatifloxacin, delafloxacin). sulfonamides (including sulfacetamide, sulfadiazine, sulfasalazine, sulfisoxazole, trimethoprim, trimethoprim-sulfamethoxazole); tetracyclines (including demeclocycline, doxycycline, minocycline, oxytetracycline, tetracycline, tigecycline) and others (including chloramphenicol, clindamycin, lincomycin, ethambutol, isoniazid, linezolid, metronidazole, mupirocin, nitrofurantoin, rifampicin, dapsone, imipenem/cilastatin), vancomycin, aztreonam), and all the above antibiotics in combination with efficacy enhancers such as avibactam, tazobactam and clavulanate.

In some embodiments, the antibiotic is chosen from penicillins, cephalosporins, monobactams, carbapenems, macrolides, lincosamides, streptogramins, aminoglycosides, quinolones (fluoroquinolones), sulfonamides, and tetracyclines.

In some embodiments, the penicillins are chosen from amoxicillin, ampicillin, bacampicillin, carbenicillin, cloxacillin, dicloxacillin—flucloxacillin, mezlocillin, nafcillin, oxacillin, penicillin G, penicillin V, piperacillin, pivampicillin, pivmecillinam, ticarcillin, and ticar.

In some embodiments, the cephalosporins are chosen from cefacetrile (cephacetrile), cefadroxil (cefadroxyl), cefalexin (cephalexin), cefaloglycin (cephaloglycin), cefalonium (cephalonium), cefaloridine (cephaloradine), cefalotin (cephalothin), cefapirin (cephapirin), cefatrizine, cefazaflur, cefazedone, cefazolin (cephazolin), cefradine (cephradine), cefroxadine, and ceftezole.

In some embodiments, the cephalosporins are chosen from cefaclor, cefamandole, cefmetazole, cefonicid, cefotetan, cefoxitin, cefprozil (cefproxil), cefuroxime, and cefuzonam.

In some embodiments, the cephalosporins are chosen from cefcapene, cefdaloxime, cefdinir, cefditoren, cefetamet, cefixime, cefmenoxime, cefodizime, cefotaxime, cefpimizole, cefpodoxime, cefteram, ceftibuten, ceftiofur, ceftiolene, ceftizoxime, ceftriaxone, cefoperazone, and ceftazidime.

In some embodiments, the cephalosporins are chosen from cefclidine, cefepime, cefluprenam, cefoselis, cefozopran, cefpirome, and cefquinome.

In some embodiments, the cephalosporins are chosen from ceftobiprole and ceftaroline.

In some embodiments, the cephalosporins are chosen from cefaclomezine, cefaloram, cefaparole, cefcanel, cefedrolor, cefempidone, cefetrizole, cefivitril, cefmatilen, cefmepidium, cefovecin, cefoxazole, cefrotil, cefsumide, cefuracetime, and ceftioxide.

In some embodiments, the monobactam is aztreonam.

In some embodiments, the carbapenems are chosen from imipenem, imipenem/cilastatin, doripenem, meropenem, and ertapenem.

In some embodiments, the marcolides are chosen from azithromycin, erythromycin, clarithromycin, dirithromycin, roxithromycin, surlid, and telithromycin.

In some embodiments, the lincosamides are chosen from clindamycin and lincomycin.

In some embodiments, the streptogramins are chosen from pristinamycin and quinupristin/dalfopristin.

In some embodiments, the aminoglycosides are chosen from amikacin, gentamicin, kanamycin, neomycin, netilmicin, paromomycin, streptomycin, and tobramycin.

In some embodiments, the quinolones are chosen from flumequine, nalidixic acid, oxolinic acid, piromidic acid, pipemidic acid, and rosoxacin.

In some embodiments, the quinolones are chosen from ciprofloxacin, enoxacin, lomefloxacin, nadifloxacin, norfloxacin, ofloxacin, pefloxacin, and rufloxacin.

In some embodiments, the quinolones are chosen from balofloxacin, gatifloxacin, grepafloxacin, levofloxacin, moxifloxacin, pazufloxacin, sparfloxacin, temafloxacin, and tosufloxacin.

In some embodiments, the quinolones are chosen from besifloxacin, clinafloxacin, gemifloxacin, sitafloxacin, trovafloxacin, and prulifloxacin.

In some embodiments, the sulfonamides are chosen from sulfamethizole, sulfamethoxazole, sulfisoxazole, and trimethoprim-sulfamethoxazole.

In some embodiments, the tetracyclines are chosen from demeclocycline, doxycycline, minocycline, oxytetracycline, tetracycline, and tigecycline.

In some embodiments, the composition further comprises and efficacy enhancer and an antibiotic. In some embodiments, the efficacy enhancer is chosen from avibactam, tazobactam and clavulanate.

In some embodiments, the antifungals are chosen from imidazoles (such as miconazole, ketoconazole, clotrimazole ,econazole, bifonazole, butoconazole, fenticonazole, isoconazole, oxiconazole, sertaconazole, sulconazole, tioconazole, and griseofulvin); triazoles (such as fluconazole, itraconazole, isavuconazole, ravuconazole, posaconazole, voriconazole, and terconazole); thiazoles (such as abafungin); allylamines (such as terbinafine, amorolfine, naftifine, and butenafine); echinocandins (such as echinocandins, anidulafungin, caspofungin, and micafungin); amphotericin B, and azole antifungals.In some embodiments, the antifungal is amphotericin B or nystatin. In some embodiments, the antifungal is terbinafine, aorolfine, or flucytosine.

In some embodiments, the antifungal is miconazole or ketoconazole.

In some embodiments, the antifungal is chosen from fluconazole, itraconazole, voriconazole, posaconazole, and ravuconazole.

In some embodiments, the antiviral is chosen from micafungin, caspofungin, and anidulafungin.

In some embodiments, the antifungal is griseofulvin.

In some embodiments, the antivirals are chosen from anti-herpetic (antiherpesvirus) agents and anti-influenza agents.

In some embodiments, the anti-herpetic agents are chosen from acyclovir, brivudine, docosanol, famciclovir, idoxuridine, penciclovir, trifluridine, and valacyclovir.

In some embodiments, the anti-influenza agents are chosen from amantadine, rimantadine, oseltamivir, and zanamivir.

In some embodiments, the antivirals are chosen from acyclovir, famciclovir, penciclovir, valacyclovir, amantadine, rimantadine, oseltamivir, and zanamivir.

In some embodiments, viral bacteriophages make it possible to reduce or eliminate colonization and/or infection of humans and animals by pathogenic bacteria, including antibiotic resistant bacteria. Compared to antibiotics, in some embodiments, phages go deeper into the infected area. Antibiotics, on the other hand and in some embodiments, have concentration properties that quickly decrease as they go below the surface of the infection. The replication of phages is concentrated on the infected area where they are needed the most, while antibiotics are metabolized and removed from the body. In addition, secondary resistance does not happen among phages, but happens quite often among antibiotics. Secondary resistance is acquired and occurs when there are not enough blood drug levels. Phages, in some embodiments, provide a good choice for the treatment of drug-resistant bacteria.

In some embodiments, the viral bacteriophages are chosen from phages belonging to a family chosen from ampullaviridae, bicaudaviridae, clavaviridae, corticoviridae, cystoviridae, fuselloviridae, globuloviridae, guttaviridae, inoviridae, leviviridae, microviridae, plasmaviridae, tectiviridae.

In some embodiments, the viral bacteriophages are used as a single phage or in combination (including any other phage belonging to a family chosen from ampullaviridae, bicaudaviridae, clavaviridae, corticoviridae, cystoviridae, fuselloviridae, globuloviridae, guttaviridae, inoviridae, leviviridae, microviridae, plasmaviridae, tectiviridae, and/or others.

In some embodiments, the antihistamines are chosen from azelastine, hydroxyzine, desloratadine, emadastine, levocabastine, azelastine, carbinoxamine, and levocetirizine. In some embodiments, the antihistamines are chosen from fexofenadine, diphenhydramine, dimetane, loratadine, clemastine, chlorpheniramine, and certirizine. In some embodiment, the antihistamines are chosen from brompheniramine, chlorpheniramine, dimenhydrinate, and doxylamine.

In some embodiments, the nasal decongestants are chosen from oxymetazoline, phenylephrine, and pseudoephedrine.

In some embodiments, the active ingredients are chose from spermicidal agents, prostaglandins, and hormones.

In some embodiments, the mucoadhesive polymers are chosen from protein based polymers, polysaccharides, polyesters, polyanhydrides, polyamides, phosphorous based polymers, acrylic polymers, vinylpyrrolidone polymers, celluloses, and silicones.

In some embodiments, the mucoadhesive polymers have a mass average molecular weight above 75,000 Da. In some embodiments, the average molecular weight ranges from 100,000 to 20,000,000 Da or from 200,000 to 1,000,000 Da or from 400,000 to 700,000 Da.

In some embodiments, the mucoadhesive polymers include in general hydrophilic polymers and hydrogels. In the large classes of hydrophilic polymers, those containing carboxylic group exhibit mucoadhesive properties; these include polyvinyl pyrrolidone (PVP), methyl cellulose (MC), sodium carboxy-methylcellulose (SCMC) hydroxy-propyl cellulose (HPC) and other cellulose derivative. Hyrogels are the class of polymeric biomaterials that exhibit the basic characteristics of swelling by absorbing water, and then they interact with the mucus that covers epithelium by means of adhesion. Polymers with anionic groups include: carbopol, polyacrylates and their cross-linked modifications, polymers with cationic groups include chitosan and its derivatives and aminoethyl methacrylate or acrylate polymers.

One or more of the following basic properties a polymer indicate a good mucoadhesive profile: high molecular weight, chain flexibility, high viscosity, optimal cross-linked density of polymer, charge and degree of ionization of polymer (anion >cation >unionized), medium pH, hydration of the polymer, high applied strength and duration of its application and high initial contact time. In addition to the above factors, some physiological factors, like mucin turnover and disease status lso affect the mucoadhesion. The mucin turnover is expected to limit the residence time of the mucoadhesive agents on the mucus layer. This could detach mucoadhesives are from the surface no matter how high the mucoadhesive strength may be.

In some embodiments, the mucoadhesive system should possess an acceptable active ingredient loading capacity, good mucoadhesion, no irritancy, good feel in the place of administration, sustained drug delivery and an erodible formulation has the added advantage of not requiring retrieval after delivery of the dose. Therefore, hydrophilic polymers with good ability to sick to mucosal membranes are a good chose. They normally possess charged groups or nonionic functional groups capable of forming hydrogen bonds with mucosal surfaces. To accomplish these properties, structural characteristics such as strong hydrogen bonding groups (e.g. carboxyl, hydroxyl, amino- and sulfate groups), strong anionic or cationic charges, high molecular weight, chain flexibility, and surface energy properties favoring spreading onto mucus are sought.

In some embodiments, anionic polymers have demonstrated mucoadhesive properties related to the ability of carboxylic groups to form hydrogen-bonds with oligosaccharide chains of mucins. In some embodiments, weakly anionic carboxyl-containing polymers such as poly(acrylic acid), poly-(methacrylic acid), sodium alginate, carboxymethylcellulose and poly(maleic acid)-co-(vinyl methyl ether) are used. In some embodiments, chitosan and some synthetic polymethacrylates are cationic polymers that have mucoadhesiveness. This property has been related to their ability to interact with negatively charged mucins via electrostatic attraction and hydrophobic effects may also play a certain role. In some embodiments, chitosan derivatives relevant to pharmaceutical applications include trimethyl chitosan, glycol chitosan, carboxymethylchitosan and half-acetylated chitosan. In some embodiments, solid micro/nanoparticulate systems based on chitosan and derivatives have been the focus of several studies.

In some embodiments, compared to the charged, non-ionic polymers generally show lesser mucoadhesiveness. The specific interactions between mucin and this kind of polymers are usually very weak. In some embodiments, amphoteric polymers like as gelatin and carboxymethylchitosan, have been explored as mucoadhesive materials for pharmaceutical systems. In some embodiments, their nature of and self-neutralization of cationic and anionic charged within their structure contribute to relatively lesser mucoadhesiveness, similar to non-ionic polymers. In some embodiments, aminated derivative of gelatin has shown a considerable gastric mucoadhesion both in vitro and in vivo in rats.

In some embodiments, polyampholyte polymers displayed particular characteristics that have to be taken into consideration with regarding to their mucoadhesive and penetration-enhancing properties. In some embodiments, they exist positively charged, neutral and negatively charged, depending on dispersion pH and their specific isoelectric point. In some embodiments, the viscosity in the dispersion is minimal and increases when pH is higher or smaller that isoelectric point.

In some embodiments, the presence of inorganic salts affects the viscosity of the dispersion. In some embodiments, the mucoadhesive and penetration enhancing properties of polyampholyte-based formulations are affected by all these pH induced structural and physicochemical transformations.

In some embodiments, there is another specific class of polymers called tiomers. They are characterized by containing side chains with thiol-bearing functional groups and are obtained by conjugating conventional mucoadhesive polymers with molecules carrying thiol functionality. The presence of this kind of functional groups enables the formation of disulfide bridges (covalent bonds) with cystein rich sub-domains of mucus glycoproteins either via thiol/disulfide exchange reactions or through a simple oxidation of free thiol groups, exhibiting significantly enhanced mucoadhesive properties in comparison with conventional mucoadhesives. In some embodiments, poly(acrylic acid)/cystein, chitosan/N-acetylcystein, alginate/cystein, chitosan/thioglycolic acid and chitosan/thioethylamidine are typical polymeric thiomers. The development of novel derivatization approaches to thiolate non-ionic polymers may offer a way to improve their poor mucoadhesive performance. In some embodiments, the polymers have acrylate end groups. They are a class of mucoadhesive polymers capable of forming covalent bonds with mucins similarly to polymeric thiomers.

In some embodiments, dendrimers have displayed usefulness as mucoadhesives due to their properties and unique structure. In some embodiments, poly(amidoamine) (PAMAM) dendrimers carrying various functional groups (amino, carboxylate and hydroxyl surface groups, COOH) are chosen for mucoadhesiveness. In some embodiments, boronic acid copolymers are chosen for mucoadhesiveness. In some embodiments, copolymers of N-acryloyl-m-aminophenylboronic acid with N,N-dimethylacrylamide (e.g., up to 15 mol-% N-acryloyl-m-aminophenylboronic acid to ensure their solubility in aqueous environment) display interaction with stomach mucin and may facilitate the retention of poly(vinyl alcohol)/borax gels in mucosal lumens, mainly at pH 7.0-9.0, where their complexation with mucins is pronounced.

In some embodiments, polymers containing sugar moieties as pendant groups (synthetic glycopolymers) possess hybrid properties. With this kind of material is possible for the easy manipulation in their architecture and physicochemical properties, which can be performed through homo- and copolymerization with monomers of different nature.

For example, glycopolymers have been obtained by free-radical copolymerization of N-(2-hydroxypropyl) methacrylamide with various sugar-containing monomers such as N-methacryloylglycylglycylgalactosamine, N-methacryloylglycylglycylfucosylamine, N-methacryloylglycylglycylglucosamine, and N-methacryloylglycylglycylmannosamine. In some embodiments, fucosylamine with copolymers are chosen, e.g., to adhere selectively to the colon in vitro, and stronger adhesion was observed for copolymers containing larger quantities of sugar moieties. The inventors hypothesized that this adhesion is related to the binding of sugar-moieties of the copolymers to specific receptors present in the colonic epithelium. The adhesion of these glycopolymers to the small intestinal mucosa was less pronounced and less sensitive to fucosamine in the copolymers.

In some embodiments, considering the great number of polymers used for developing such systems, that ones derived from polyacrylic acid, such as polycarbophil and carbomers; polymers derived from cellulose, such as hydroxyethylcellulose and carboxymethylcellulose; alginates, chitosan and derivatives, lectins and their derivatives are chosen.

In some embodiments, the protein based polymers are chosen from collagens, albumins, and gelatins. In some embodiments, the albumin is conjugated to poly-(ethylene glycol).

In some embodiments, the polysaccharides are chosen from alginates, cyclodextrines, chitosans, dextrans, agarose, hyaluronic acid, starch, and cellulose.

In some embodiments, the polyesters are chosen from poly lactic acid (PLA), polyglycolic acid (PGA), poly lactide-co-glycolide (PLGA), polyhydroxybutyrate (PHB), poly(e-caprolactone), polydioxanone.

In some embodiments, the celluloses are chosen from carboxymethyl cellulose (CMC), methyl cellulose (MC), hydroxyethylcellulose (HEC), hydroxypropyl methyl cellulose (HPMC), hydroxylpropyl cellulose(HFC), ethyl hydroxyethyl cellulose (EHEC), and methyl hydroxyethyl cellulose(MHEC).

In some embodiments, the mucoadhesive polymer has one or more strong hydrogen bonding groups chosen from —OH and —COOH.

In some embodiments, the mucoadhesive polymer is chosen from high molecular weight homo- and copolymers of acrylic acid crosslinked with a polyalkenyl polyether. In some embodiments, the mucoadhesive polymer is chosen from crosslinked acrylic or methacrylic acid based polymers. For example, in some embodiments, the mucoadhesive polymer is chosen from Carbopol or Carbomer brand polymers. For example, in some embodiments, the mucoadhesive polymer is chosen from Carbopol® 934 Polymer, Carbopol® 940 Polymer, Carbopol® 941 Polymer, Carbopol® 980 Polymer, Carbopol® 981 Polymer, Carbopol® 1342 Polymer (Acrylates/C10-30 Alkyl Acrylate Crosspolymer), Carbopol® 1382 Polymer (Acrylates/C10-30 Alkyl Acrylate Crosspolymer), Carbopol® 2984 Polymer, Carbopol® 5984 Polymer, Carbopol® SC-200 Polymer (Acrylates/C10-30 Alkyl Acrylate Crosspolymer, and Carbopol® Silk 100 Polymer. In some embodiments, the mucoadhesive polymer is Carbopol® 940 Polymer.

In some embodiments, the mucoadhesive polymer is chosen from hydroxy propyl cellulose (HPC) or hydroxy propyl methyl cellulose (HPMC).

In some embodiments, the mucoadhesive polymer has an anionic charge.

Another strategy to adjust mucoadhesive properties of the system, to optimize their mechanical characteristics, to modulate their swelling behavior or to improve their biocompatibility is to use the polymers blends. New mucoadhesive blends may be obtained by mixture of pharmaceutical polymers in solid state or in solution. When two of these mucoadhesive materials are blended, their mucoadhesive properties are dependent on the strength of specific interactions occurring between both components upon hydration. When there is not the formation of insoluble polycomplexes, the specific interactions between the polymers are not very strong and the mucoadhesiveness of a system will often be intermediate between the adhesiveness of each individual component. Interpolymer complexes like as poly (carboxylic acids) and non-ionic polymers in solutions via hydrogen bonding results in formation of novel polymeric materials-interpolymer complexes. These materials can potentially be used for design of novel mucoadhesive dosage forms.

In some embodiments, the mucoadhesive polymer is a blend of two or more mucoadhesive polymers noted herein. For example, in some embodiments, the blend comprises from 50 to 90% by weight of the blend of Carbopol or Carbomer brand polymers, such as those noted herein; and from 10 to 50% by weight of the blend of hydroxy propyl cellulose (HPC) or hydroxy propyl methyl cellulose (HPMC).

In some embodiments, colloidal semi-solid systems, liquid crystalline mesophases and hydrogels dispersions are used, which can increase the contact time between preparation and mucous membrane after they undergo in situ gelation. Thermodynamically stable and isotropic liquid systems such as microemulsions allow the incorporation of bioadhesive molecules, such as polycarbophil.

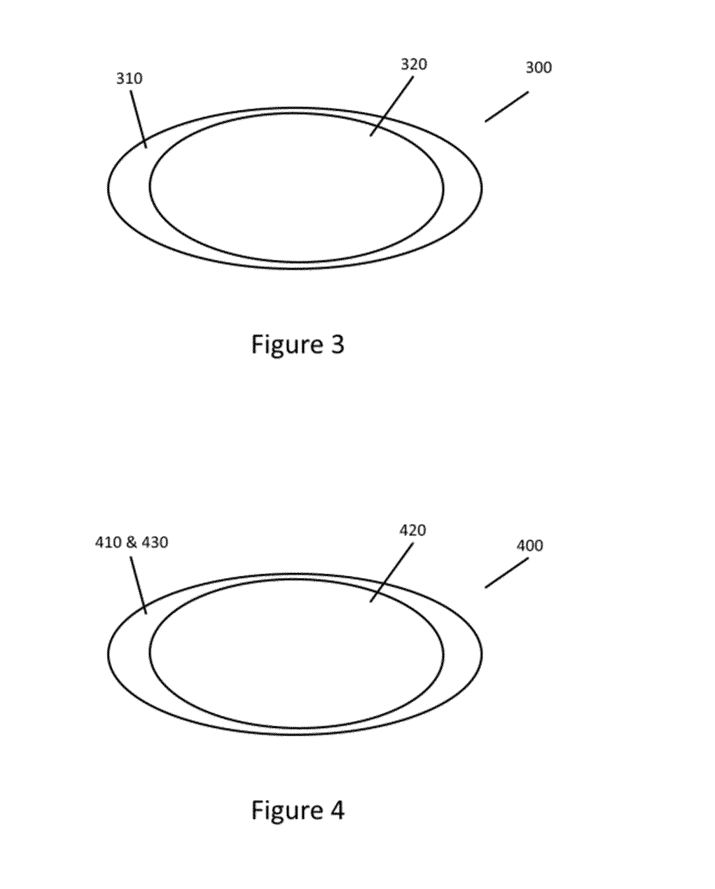

FIG. 3 illustrates an embodiment. The pharmaceutically acceptable composition 300 has a mucoadhesive polymer 310 coating, dissolving or dipersing an active ingredient 320.

In some embodiments, the mucoadhesive polymer is crosslinked (at least partially or fully). In some embodiments, crosslinking results from exposure to water, e.g., during mucoadhesion, or a crosslinking agent.

In some embodiments, the composition further comprises one or more other ingredients. In some embodiments the one or more other ingredients are coated along with the active ingredients. In some embodiments, the one or more other ingredients are dispersed in or dissolved in the mucoadhesive polymer coating, dissolving or dispersing the active ingredient.

FIG. 4 illustrates an embodiment. The pharmaceutically acceptable composition 400 has a mucoadhesive polymer 410 coating an active ingredient 420. Mucoadhesive polymer 410 has dispersed or dissolved therein active ingredient 430.

In some embodiments, active ingredient 430 is the same as active ingredient 420. In some embodiments, active ingredient 430 is the same as active ingredient 420. For example, active ingredient 430 is chosen from anti-inflammatory agents, such as those described above, and active ingredient 420 is chosen from antimicrobial active agents, such as those described above.

Although the one or more other ingredient is described as an active ingredient, the one or more other ingredient is not so limited and is, in some embodiments, chosen from the other ingredients listed herein.

For example, in some embodiments, the composition further comprises a delivery enhancer. In some embodiments, the delivery enhancer is chosen from gap junction openers, p-glycoprotein pump inhibitors.

In some embodiments, the p-glycoprotein pump inhibitors are chosen from polysorbate 80. In some embodiments, the p-glycoprotein pump inhibitors are chosen from quinidine, verapamil, and amiodarone.

In some embodiments, the gap junction opener is trimethylamine or edta (ethylenediaminetetraacetic acid).

In some embodiments, the composition further comprises water. In some embodiments, the composition further comprises a liquid pharmaceutically acceptable carrier, such as ethanol, oils, and water.

In some embodiments, the composition comprises one or more excipients, gelling agents, viscosifying agents, and pH modifiers.

In some embodiments, the pharmaceutically acceptable composition is embedded in a solid pharmaceutically acceptable carrier that is optionally coated with a second mucoadhesive polymer, which is optionally cross linked (in part or whole). In some embodiments, the second mucoadhesive polymer is different than the mucoadhesive polymer coating the active ingredient of the pharmaceutically acceptable composition.

In some embodiments, the amount of the pharmaceutically acceptable composition ranges from 10 to 90 percent by weight (w/w %) relative to the weight of the pharmaceutically acceptable composition and solid pharmaceutically acceptable carrier. In some embodiments, the amount ranges from 20 to 80 w/w % or from 30 to 70 w/w %.

In some embodiments, the composition has a form chosen from gels, lyophilized powers, powders, suspensions, and solution. In some embodiments, the composition has a form chosen from tablets, films, tampons and rings to foams, semisolids, sponges, creams, gels, solutions, ointments, ovules, soft gelatin capsules, pessaries, douches, suppositories and microemulsions.

In some embodiments, the composition is sterilized or pasteurized.

In some embodiments, the coating of the coated particles forms a gel having properties of muco-adhesion. In some embodiments, such coatings are makeable from polymers, such as acrylic based polymers, like methacrylic acid co-polymers, which can bind to the mucous layer due to its unique pH/solubility characteristics and ionic/hydrophobic interactions.

In some embodiments, the muco-adhesive gel may also contain other delivery enhancers to help with delivery of active ingredients, such as anti-inflammatory or antimicrobial active agents, to the bio-film of the mucosae. In some embodiments, the delivery enhancer is chosen from gap junction openers (such as EDTA etc.) and p-glycoprotein pump inhibitors (such as polysorbate 80 etc.). In some embodiments, an anti-inflammatory is dissolved or dispersed in the gel matrix along with one or more delivery enhancers.

After the bio-adhesive film that contains the various active components at an effective concentration above the MIC is applied onto the bio-film, the active ingredient(s) is absorbed into the bio-film and the patient's tissue under the bio-film. This absorption at the muco-adhesive gel/bio-film interface lowers the local concentration of the active ingredient(s). This creates a concentration gradient within the muco-adhesive gel, with the highest concentration farthest away from the bio-film and the lowest concentration at the interface. This concentration gradient promotes diffusion of the active (antibiotic/anti-infective and/or anti-inflammatory from the zones of highest concentration to the lowest concentration at the interface. The diffusion promotes replenishment of the absorbed active ingredient at the interface so that the MIC is maintained for the desired time to disrupt the bio-film and destroy the organisms forming the bio-film.

In some embodiments, the above mixture of mucoadhesive polymer, anti-inflammatory and delivery enhancers may be sterilized or pasteurized in a single use container such as a vial or in a form/fill/seal container, to reduce the chances of introducing bad organisms when applying the gel. In some embodiments, the contents of the vial or other suitable container could be used to mix with a solution of the antimicrobial/antibactierial/antiinfective agent or re-constitute the antimicrobial/antibactierial/antiinfective agent which is available as a lyophilized cake or powder.

In some embodiments, the composition is administered to the nasal mucosa for nasal administration.

In some embodiments, the composition is in the form of a mucosa-adhesive nanoparticle formulation suitable for the treatment of diseases of the nose or sinuses (such as rhinosinusitis).

In some embodiments, the composition is in the form of a bioadhesive and biodegradable and formulation that make it possible to provide sustained release of antimicrobials, bacteriophages and/or anti-inflammatory medications for inactivation of biofilms and the treatment of rhinosinusitis and other infections.

In some embodiments, the composition is suitable for nose and sinus mucosa. In humans and other animal species, the main functions of the nasal cavity are breathing and olfaction, and it provides a protective activity that is to filter, heat and humidity the inhaled air before reaching the lowest airways. The layer of mucus and hairs are responsible for trapping inhaled particles and pathogens. The nasal respiratory mucosa is a membrane formed by numerous microvilli lined by a pseudostratified columnar epithelium underlined with a very rich vascularization. It is supported on the collagen basement membrane, lamina propria, which is richly supplied, with blood vessels, nerves, glands and immune cells. The epithelium is mainly composed of basal cells, ciliated column cells, non-ciliated column cells and globlet cells. It is believed that basal cells are responsible for assisting the adhesion of the basal membrane; while columnar cells responsible for most of the epithelium. Their apical surface contains microvilli, which considerably increase the surface area of the respiratory epithelium, and the globlet cells secrete mucin, contributing in part for the production of the mucus layer. This nasal mucus layer has only 5 μm thick and it consists of 95% of water, 2.5-3% of mucin and 2% of electrolytes, proteins, lipids, enzymes, antibodies, sloughed epithelial cells and bacterial products.

The nasal passages are a very attractive route for a wide range of therapeutic compounds. Therefore, compared with other mucosal application sites, the nose has many advantages such as high vascularization; fairly wide absorption area; porous and thin endothelial basement membrane of the nasal epithelium; potential alternative route for systemic delivery of small drugs not absorbed via oral route.

However, the nasal route has some limits. These barriers include relatively rapid removal of the drug from the site of deposition, by the elimination mechanisms of mucociliary clearance, enzymatic degradation in mucus layer, low permeability of the nasal epithelium due to nasal pathology. In some embodiments, the composition is suitable for nasal administration of medicines for the treatment of a topical nasal disorder such as rhinosinusitis. In some embodiments, the topical disorder is treatable using an antihistamine and an anti-inflammatory agent (such as those noted above, e.g., a corticosteroid) for rhinosinusitis; an antibiotic and optional anti-inflammatory agent (such as those noted above, e.g., a corticosteroid) for chronic rhinosinusitis; or a nasal decongestant for cold symptoms.

Mucoadhesive systems constitute a strategy that can improve nasal drug absorption by the maintenance of the formulation adjacent to the nasal mucosa for an extended time period and hence increase bioavailability of the drug. In some embodiments, solution and suspension sprays are used, and in some embodiments, lipid emulsions, mi/nanoparticles, liposomes, gels and films (which have to be applied directly to the areas of the sinus mucosa) are used. In some embodiments, powders or suspensions are used. Therefore, various excipients have been used in the preparation of such formulations with mucoadhesive characteristics, among them mucoadhesive polymers and other gelling/viscosifying agents may be highlighted.

In some embodiments, the antimicrobials are chosen from those for treating rhinosinusitis also include bacteriophages that have the potential to reduce or eliminate colonization and/or infection and/or biofilms by pathogenic bacteria, including antibiotic resistant bacteria. Bacteriophages (phages) are bacterial viruses that infect and lyse bacterial cells. Phages have the ability to diffuse through the biofilm matrix, facilitating phage access to biofilm-bound cells, which are subsequently infected and destroyed by the phage. Phages have been shown in numerous studies to be effective against biofilms, including biofilms of S. aureus. Cocktails of S. aureus specific phage (CTSA) have been shown to be effective against biofilms of S. aureus clinical isolates obtained from CRS patients in vitro. Clinical administration of phage has also been shown to be safe when applied orally, as well as topically, to the ear, external wounds/venous stasis, and leg ulcers.

In some embodiments, the active agent is a net drug particle that is insoluble and available in particulate form of micronized size and/or of bacteriophage. In some embodiments, the drug and /or phage is encapsulated in a biodegradable polymer shale or matrix. In some embodiments, these particles are coated with a mucoadhesive polymer that has an affinity to the inflamed sinus mucosal surface. In some embodiments, Mucosal coatings on particles are hydrophobized chitosan or alginates by a fatty chain to increase their adhesion to the particle surface and form a water insoluble polymer. In some embodiments, other polymers are crosslinked polyacrylic acid or polymethacrylic acid and copolymers with alkyl acrylates or acryl amide. Such compounds are commercially available such as Carbopol series. In some embodiments, the drug particles are coated with a mucoadhesive polymer, loaded in a polymer matrix that is coated with a mucoadhesive layer or absorbed in the mucosdhesive polymer matrix. Other relevant polymers are copolymers of maleic anhydride with octadecene or with ethyl vinyl ether (Gantrez) where upon hydrolysis forms a polycarboxylic acid mucoadhesive. These carboxylic acid containing polymers may be mixed with a polyol such as hydroxy propyl cellulose or hydroxyl propyl-methylcellulose. In some embodiments, the particles can be pre-coated with a lipid molecule such as fatty acid, alcohol or amine or a biodegradable polymer and coat on top with mucoadhesive polymer. The mucoadhesive coating is tailored to provide long retention time onto mucosal tissue. Such mucoadhesive polymers may include physical salts of fatty acids with chitosan or acrylate polymers containing amino groups or carboxylic acid containing polymers such as hyaluronic acid, alginate or acrylic acid polymers mixed with fatty amine. Alternatively, the charged polymers are modified by conjugation to lipids such as fatty chains, phospholipids and polyethylene glycol. The fixation of the mucoadhesive polymer onto the particle surface is obtained by crosslinking with a bifunctional molecule such as propane-dialdehyde or a polyaldehyde such as oxidized dextran or cellulose.

In some embodiments, the composition comprises coated particles with specific affinity to the mucosal surface of the sinus so that the particles stick to the surface when administered, e.g., via nasal spray or by direct cannula when the product is delivered directly to the sinus cavity. The specific mucoadhesion surface is designed to retain the particles that reach the mucosal surface of the sinuses.

In some embodiments, the composition comprises nano and micron size particles (0.05-15 microns) that can be loaded with an active ingredient having a mucoadhesive surface to enhance their adhesion and retention with the sinus mucosa. These can provide a suitable vehicle for extended drug delivery over a certain period of time (extending from hours to several days). The drugs relevant for this application are those that are sufficient to treat rhinosinusitis. These includes: anti-inflammatory agents (such as those noted above, e.g., corticosteroids, including dexamethasone, mometasone, triamcinolone, etc.), antimicrobials (such as those noted above, e.g., antibiotics and antifungals e.g. penicillins, cephalosporins, quinolones, aminoglycosides, amphotericin B, etc.) and combinations thereof

In some embodiments, the composition is in the form of a mucosa-adhesive soluble or dispersion formulations for the treatment of diseases of the nose and sinuses (such as rhinosinusitis).

In some embodiments, the composition is a sterilized or pasteurized ready-to-use solution or dispersion containing a mixture of mucoadhesive polymer, anti-inflammatory, delivery enhancers (such as EDTA and Polysorbate 80 etc.) which may be mixed with antibiotics (selected for their action on the infecting bacteria) in the clinic which is going to administering the mixture to the patient.

In some embodiments, the dispersion of the active ingredients (anti-inflammatory agents and antimicrobial/antifungal/antiinfective active agents/bacteriophage, etc.) occurs following a concentration gradient, with the highest concentration farthest away from the bio-film and the lowest concentration at the interface. The low concentration (at the surface of the bio-film) is created by the absorption/diffusion of the active ingredient (antibiotic and/or anti-inflammatory) and optional absorption enhancers (like EDTA) into the bio-film first and then into the patient's tissue. In some embodiments, the gradient is facilitated by absorption enhancers, such as EDTA, surfactants, bile salts, phospholipids, chitosan, etc.

In some embodiments, the composition is in compartments of a kit to minimize introducing bad bacteria to the site of the infection/inflammation. In some embodiments, it is easier for the staff in the clinic doing the nasal delivery to select the desired anti-biotic and prepare the solution/dispersion for nasal administration.

The synergistic combination of the antibiotic and the anti-inflammatory with the muco-adhesive polymer and delivery enhancers makes it possible to achieve significant advantage over the current systems.

In some embodiments, the composition comprises an active ingredient chosen from anti-inflammatory agents (such as corticosteroids, including dexamethasone, mometasone, triamcinolone, etc.), antimicrobials (such as antibiotics and antifungals, e.g., penicillins, cephalosporins, quinolones, aminoglycosides, amphotericin B, etc.) and combinations thereof

In some embodiments, the muco-adhesive polymer is chosen from those that are safe for oral consumption (i.e., GRAS), as this polymer may flow down the gastrointestinal GI system to the stomach from the nasal cavity. In some embodiments, use of polymers that are not GRAS may contribute to undesirable side-effects. In some embodiments, a methacrylic co-polymer system is chosen. And in some embodiments, chosen is a methacrylic co-polymer, such as those available as Kollicoat (from BASF) MAE-30D, and the anti-inflammatory would be chosen from a group that does not degrade at temperatures in the range of 110 to 130 C and sterilized (i.e., stable at up to ˜120 C for 15 minutes).

In some embodiments, the composition is in the form of a mucosa-adhesive nanoparticle formulation for the treatment of diseases other than the nose and sinuses.

In some embodiments, the composition provides coated particles with affinity to the other mucosal surfaces in the body, different from the nasal and sinus mucosa: including the oral mucosa, rectal mucosa, vaginal mucosa, ocular mucosa.

The mocosal membrane is within the main administration site for bioadhesive preparations. It acts as a semi-permeable barrier system where water, nutrients, gases, selected small molecules and ions are allowed to diffuse through. They are characterized by an epithelial layer whose surface is covered by mucus that contains glycoproteins, lipids, inorganic salts and 95% water by mass, making it a highly hydrated system. Mucin is the most important glycoprotein of mucus and is responsible for its structure, protecting and lubricating the epithelium and other additional functions depending on the epithelium covered. There are two types of mucin, membrane-bound and secreted (soluble) biomacromolecules forming a fully-hydrated viscoelastic gel layer (mucus).

Soluble mucin possesses high-molecular weight (0.5-40 MDa) composed of 500 kDa sub-units linked together by peptide linkages and intramolecular cystein—cysteine disulfide bridges. The thickness of mucus is approximately of 50-450 μm in the stomach to less than 1 μm in the oral cavity. Therefore, this mucus gel is a dynamic system reformed continuously through the secretion of mucins from the goblet cells.

The market for mucoadhesive therapeutic systems is expanding rapidly. They constitute attractive and flexible dosage forms due to the possibility of various administration routes (buccal, gastrointestinal, vaginal, ocular, rectal and nasal) and their composition is dependent of the characteristics of the administration site. Therefore, it is very important to understand the particularities of the mucosal places where bioadhesive systems are administered.

Oral Mucosal Cavity

The oral mucosal cavity possesses a relatively permeable mucosa with a rich blood supply. Robust, it shows short recovery times after stress or damage, and it is tolerant to potential allergens, being a very attractive and feasible site for drug delivery.

In comparing the structure oral mucosa to the gastrointestinal tract, a major difference emerges in the organization of the epithelium. In this context, the lining of the stomach and the small and large intestine consist of a simple epithelium composed of only a single layer of cells. Oral mucosa is covered by a stratified epithelium composed of cells, which show various patterns of maturation between the deepest cell layer and the surface. Drug delivery across this stratified epithelium offers a safer method of drug utilization, avoiding the presystemic metabolism in the gastrointestinal tract. In addition, drug absorption can be promptly terminated in cases of toxicity by removing the system from the buccal cavity.

Oral mucosa has two permeability barriers. The intercellular spaces and cytoplasm are essentially hydrophilic in character and the cell membrane, rather lipophilic in nature with a low partition coefficient. Thus, the intercellular spaces act as the major barrier to permeation of lipophilic compounds and the cell membrane acts as the major transport barrier for hydrophilic compounds. Therefore, the drug transport in the oral mucosa, and many others mucosae, may involve a combination of the paracellular and the transcellular routes.

Oral mucosal drug delivery is classified into sublingual delivery (systemic delivery of drugs through the mucosal membranes lining the floor of the mouth), buccal delivery drug administration through the mucosal membranes lining the cheeks or buccal mucosa, and local delivery (where the drug is delivery into the oral cavity). Intraperiodontal pocket drug delivery is a special category where the drug delivery happens in a specific site, within the periodontal pocket, being generally used for treatment of periodontitis.

Therefore, oral or gastrointestinal mucosal delivery systems can be mucoadhesives, which interact with the mucincoated epithelial or tooth surfaces by bioadhesion, producing sustained effect, ensuring the formulation retention on the place. The use of mucoadhesive platforms is useful to prolong the drug delivery in the oral cavity and the gastrointestinal tract, and to improve the therapeutics.

In some embodiments, the composition is administered to the oral mucosa or a tooth surface in the oral cavity.

Rectal mucosa

Among the various body systems, the digestive has the important function of mastication, ingestion and absorption of food and elimination of waste. The rectum is part of that system and is located at its end portion. The volume, length and diameter of the rectum change during the body development. The adult rectum is formed by the distal large intestine, and has length of about 15-19 cm and diameter of 15-35 cm. In the last 4-5 cm (proximal part of the anal canal), there is a transition that changes columnar epithelium to stratified squamous epithelium. The surface absorption is only 1/10000 of upper gastrointestinal (GI) tract. The rectum does not have microvilli, but the mucous membrane is present. The absorption of water and sodium in the rectum is insignificant, and the primary mechanism from rectal drug delivery is the passive transport.

The third most lethal cause of cancer death is the colorectal cancer. Nowadays, the treatment after surgery for this kind of diseases is chemotherapy, radiotherapy and tumor resection. The treatment often uses injection or oral administration. The rectal is not the first choice route but has many benefits to the patient, including local or systemic effects. The oral route has the disadvantage of the hepatic first-pass effect.

When administered by intravenously route, some anticancer drugs can damage the vein in which is injected. Therefore, rectal mucoadhesive systems constitute an alternative to overcome these problems. Moreover, systemic treatment using the rectal route is also a great alternative for treating children, especially the ones that are unable to swallow any drug, patients who are mentally disturbed, unconscious or unable to tolerate oral medication, when oral administration is no feasible. This route can rapidly achieve systemic effect and is an effective route of administration for various compounds like analgesics, sedatives, anticonvulsivants, anti-inflammatory drugs, antibiotics and antiepiletics as well.

Traditional rectal dosage forms, like as the suppositories, have the disadvantage of softening or melting in the rectum which gives a discomfort feeling to the patients.

Furthermore, they have characteristics of sustained release for drug. New dosage forms containing strategies to overcome these problems are sought and suppositories and enemas with mucoadhesive properties have been proposed. The liquid mucoadhesive suppository is desirable, e.g., to form a gel at a body temperature. It can have a suitable bioadhesive force and suitable gel strength. The ideal suppository or enema should have mucoadhesive characteristics to stay in the rectum and remain there for an appropriate period of time. Thermosensitive liquid dispersions are easily administered into the anus and operate as mucoadhesive to the rectal mucosal tissues.

In some embodiments, the active ingredient is chosen from analgesics, sedatives, anticonvulsivants, anti-inflammatory drugs, antibiotics and antiepiletics. In some embodiments, the composition is in the form of an enema or a suppository.

Vaginal Mucosa

The vagina plays a major role in reproduction, being an important organ of the reproductive tract. It is a muscular, strong tubular, positioned between the rectum, bladder and urethra with dimensions range from 8.4 to 11.3 cm in length and 2.1 to 5.0 cm in diameter and a slightly S-shaped fibromuscular collapsible tube connected the cervix (the opening of the uterus) and the vulva (the external genitalia). The surface of the vagina is composed of numerous folds (wrinkles), which provide distensibility, support and an increased surface area of the vaginal wall. The vagina has an excellent elasticity because of the presence of smooth elastic fibers in the muscular coat. The epithelial layer is a non-cornified, stratified squamous epithelium and its thickness is dependent on age as well as different stages of the cycle. With hormonal activity, the vaginal epithelium increases in thickness, is highest in the proliferative stage, and reaches the highest glycogen content during ovulation.

The vaginal tissues do not possess any gland, but secrets a large amount of fluid, produced from cervical secretion and transudation from the blood vessels with desquamated vaginal cells and leucocytes mainly, as well as the secretions from the endometrium and fallopian tubes. Thus, cervico-vaginal mucus is a gel layer consisting by a mixture of 95% water, 1%-2% secreted mucin. Trace amounts of other components like lactic acid, lipids, salts, proteins, enzymes, enzymatic inhibitors, carbohydrates, amino acids, alcohols, hydroxylketones, aromatic compounds and transudates through the epithelium, cervical mucus exfoliating epithelial cells, secretions of the Bartholin's glands, leukocytes, endometrial and tubal fluids are present as well. The vaginal fluids possesses special characteristics like cervical mucus presence, it has impacts on drug delivery in the vagina in various ways. The presence of this physiological fluids may alter the characteristics of a vaginal product, which can reduce the overall efficacy of the drug substance, increase leakage, and decrease drug residence time at the target tissue. Moreover, these fluids result in product dilution and can alter drug dissolution, ultimately playing a role in the success of getting the drug to its target site.

Vaginal is a non-invasive route of administration. Compared with other mucosal application sites, the vagina has many advantages such as: the avoidance of hepatic first-pass metabolism; a fall in the incidence and severity of gastrointestinal side effects; avoidance of the inconvenience caused by pain, tissue damage and risk of infections which are associated with parenteral routes; and ease of self-insertion and removal of the dosage form is possible. However, several drawbacks should be addressed during the design of a vaginal formulation. These include cultural sensitivity, personal hygiene, gender specificity, local irritation and influence of sex and the tract's self-cleansing action. Further, considerable variability in the rate and extent of absorption of the drug is administered vaginally observed by changes in thickness of the vaginal epithelium.

Vaginal mucosa has been traditionally used either to provide women a therapy for local disorders, for the administration of locally acting drugs such as antifungal, antimicrobial, antiprotozoal, antiviral agents, spermicidal agents, prostaglandins, hormones, vaccines, anti-inflammatory, peptides/proteins, DNA plasmids or as an alternative route for systemic administration. Because of these advantages, the interest for vaginal mucosa drug delivery systems has increased considerably. It is possible to ensure a sufficiently long interaction of drug delivery systems with the vaginal mucosa, offering a broad field of applications and using various different dosage forms ranging from solid devices like tablets, films, tampons and rings to foams, semisolids, sponges, creams, gels, solutions, ointments, ovules, soft gelatin capsules, pessaries, douches, suppositories and microemulsions. Therefore, the vaginal mucosa has been used to administer mucoadhesive systems containing active agents for contraception, treatment and/or prevention of viral infections, treatment of vaginal infections, relief of vaginal itch, vaginal cleansing, and enhancement of vaginal lubrication.

In some embodiments, the active ingredient is chosen from antifungal, antimicrobial, antiprotozoal, antiviral agents, spermicidal agents, prostaglandins, hormones, vaccines, anti-inflammatory, peptides/proteins, DNA plasmids, active agents for contraception, treatment and/or prevention of viral infections, treatment of vaginal infections, relief of vaginal itch, vaginal cleansing, and enhancement of vaginal lubrication. “ ” composition is in the form of a tablet, film, foam, cream, gel, solution, ointment, ovules, soft gelatin capsules, pessaries, douches, suppositories and microemulsions. In some embodiments, the composition is coated on a tampon, sponge, diaphragm, or ring for the vagina.

In some embodiments, the composition treats vaginal itch, provides contraception, lessens vaginal dryness, or cleans a vagina.

Ocular Mucosa

Ophthalmic dosage forms have been one of the most interesting, mainly when thinking of ophthalmic illness, being preferred over the systemic administration. The conventional ophthalmic drug delivery dosage forms are eye drops (solutions or suspensions) and semisolids, such as ointments. Both should form a thin film over outer layer of the sclera. The greatest disadvantage of ocular route is the low bioavailability of the active compounds after administration, mainly because the tear dilutes those substances and washes them away.

Nowadays, there are some techniques used to overcome this drawback like using ocular inserts, in situ gelling polymers, micro/nanoparticles, liposomes, prodrugs and mucoadhesive preparations. This kind of formulations presents prolonged contact time with the local tissue. More specifically, besides the known about the conjunctival globlet cells and mucoadhesive polymers dates before the recognition of mucoadhesion.

The ocular globe located within the bony orbital cavity of the head, constituting an isolated and highly protected organ. The vascularized mucous membrane called conjunctiva covers the anterior surface of the globe with exception of the bulbar conjunctiva (cornea) and it also covers the internal surface of the eyelids (palpebral conjunctiva). The epithelium of the conjunctiva is continuous, multilayered, nonkeratinized, and columnar. It contains five to seven layers, covering the highly vascularized substantia propria. The cornea is avascularized and transparent, constituted by the arrangement of five layers of cells. There is the tear film covering the bulbar and palpebral conjunctiva and acts as a wetting agent, reducing the interfacial tension between cornea and tears, lubricating and protecting the underlying epithelial cells. This film is composed of three layers. A thin lipid monolayer (the outermost portion) is responsible to reduce evaporation and to provide a continuous covering of the underlying portions. An aqueous layer is the middle portion and constitutes more than 95% of the total volume and contains electrolytes and proteins. The basal tear layer (inner) is composed mostly of mucus glycoproteins and coats the epithelial microvilli. Cornea and conjunctiva are coated with a thin layer of mucin, secreted by approximately 1.5 million of globlet cells located on the conjunctiva surface and spread over the surface of the eye. Therefore, there is a tightly packed of mucin molecules on the corneal mucosal surface which becomes less densely packed as one moves outward from the epithelial surface. Despite the mucus layers covering the cornea are thin; they are thick enough to occur a significant interpenetration with the bioadhesive material. Moreover, the residence time of mucin in the conjunctival site is long and its production is very rapid to compensate for the loss due to digestion, bacterial degradation, and solubilization of mucin molecules.

There are precorneal elimination factors that reduce the contact time of the formulation with the corneal surface such as: drainage of instilled solutions; lacrimation and tear turnover; drug metabolism; tear evaporation; nonproductive absorption and adsorption; possible binding by lacrimal proteins. In this sense, viscous liquids, semisolids, inserts, and micro/nanoparticulates have been proposed and using different types of mucoadhesive materials. The aim is to provide long times of contact of ophthalmic drug delivery systems with the absorbing tissues, establishing noncovalent bonds with the mucin layer coating the corneal-conjunctival epithelium. For example, in this decade, liquid crystalline nanoparticles were developed employing some polymers as poloxamer 407, presenting mucoadhesive properties that denoted great bioavailability. However, these mucoadhesive systems may present high viscosity, which leads to patient discomfort. Mucoadhesive nanoparticles were developed for ocular sustained drug release, containing chitosan. Their formulation improved the retention time, the ocular availability also it presented sustained release, thus helping to reduce the dose and the frequency.

The compositions are makeable, e.g., by one of ordinary skill in the art.

Additional strategies and composition for any mucoadhesive therapeutic system.

The different mucoadhesive drug delivery systems may be grouped into twelve categories: tablets, gels, viscous solutions, pessaries, lozenges, solid inserts, wafers, films, micro- and nano-particulates, suspensions, in situ gelling systems and sprays.

In some embodiments, the composition further comprises polymeric excipients to prepare these formulations and play a role in their mucoadhesion. In some embodiments, some mucoadhesive polymers increase the dosage form residence time at the site of administration, enhance drug permeability through the epithelium by modifying the tight junctions between the cells and inhibit enzymatic degradation of active agent.

In some embodiments, the composition further comprises permeation enhancers to increase the membrane permeation rate or drug absorption rate by overcoming the restriction of the paracellular transport pathway. They are substances added to a pharmaceutical formulation to increase the bioavailability of drugs with poor membrane permeation properties, without damaging the membrane and causing toxicity. In some embodiments, the mucosal permeation enhancers are chosen from bile salt, surfactants, or an azone. In some embodiments, bioavailability of peptide drugs has been increased from approximately 5% to 30-40%. In some embodiments, chitosan is an efficient and well established enhancer of absorption across mucosal epithelia.

### EXAMPLES

Methods

Chitosan labelling with fluorescent dansyl chloride

40 g of chitosan (0.248 mole of primary amine groups) were labelled with dansyl chloride by suspending chitosan in 250 ml extra dry dichloromethane containing 670 mg dansyl chloride (1% mol/mol free amine groups) stirred overnight at room temperature. Labelled chitosan was separated by filtration followed by washing with 3×300 ml dichloromethane and 2×300 ml ethanol and evaporated to dryness.

Coating of Antifungal Particles

Chitosan-20%, 10% and 5% w/w Coating on Antifungal Agent

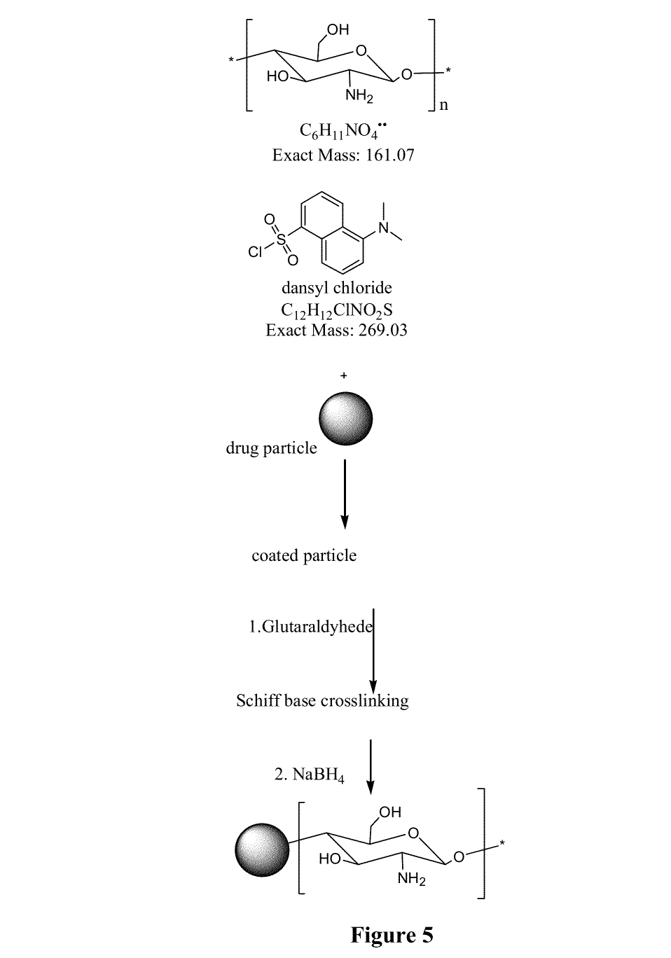

200 mg of fluorescent labeled chitosan dissolved in 17.5 ml 1.2% acetic solution in 20 ml vail using slight heating (30-40° C., 48 hr) then 800 mg of antifungal agent (Amphotericin B) particles were added and stirred in hood until complete dryness (48-72 hr). The resulted bulky coated antifungal agent was ground using mortar and pestle to form a powder. Similar formulations having 20% w/w, −10% w/w, and −5% w/w loading were prepared. See Scheme 1 (FIG. 5).

Coating Crosslinking by gluteraldehyde

700 mg of coated particles at section 2.2.2.1 were suspended in 25 ml of 1% sodium bicarbonate solution and glutaraldehyde was added in molar ratio 1:0.25 glutaraldehyde to chitosan (87 μl of 25% GA in DDW) diluted into 10 ml (1% sodium bicarbonate solution) were added in droplet manner into 2 times (5+5 ml), half hour each time with delay of 1 hour between each time and then the reaction stirring was continued for 24 hr at room temperature*. Then samples were centrifuged for 90 min at 4000 rpm and the solution was filtered using 0.45 μm filtration paper and 50 ml DDW were added and tubes were vortexed vigorously (to take away salts) and centrifuged for 60 min at 4000rpm, followed by filtration and drying overnight. Samples ShF-8-36A-C. See scheme 1 (FIG. 5).

*The same manner was used for the other ratios: ShF-8-35B, 10% (43.5 μl of 25% GA in DDW) and ShF-8-35C, 5% (21.7 μl of 25% GA in DDW).

Imine bonds reduction by sodium borohydride

350 mg of samples prepared at section 2.2.2.2, ShF-8-36A were suspended in 30 ml of DDW, to the suspension sodium borohydride were added at 2:1 ratio (NaBH₄: amine group in chitosan, 33 mg, and then samples were stirred at room temperature for 36 h**. The samples were centrifuged for 90 min at 4000rpm and the isolated solution was filtered using 0.45 μm filtration paper and samples were collected to centrifuge tubes and 50 ml DDW were added and tubes were vortexed vigorously and centrifuged for 60 min at 4000rpm, followed by filtration and drying overnight in hood. The bulky coated drug particles, including antifungal agent was ground to powder. See scheme 1 (FIG. 5).

**The same manner was used for the other ratios: 10% (16.5 mg of NaBH₄) and 5% (8.25 mg of NaBH4).

Dansyl ethylenediamine

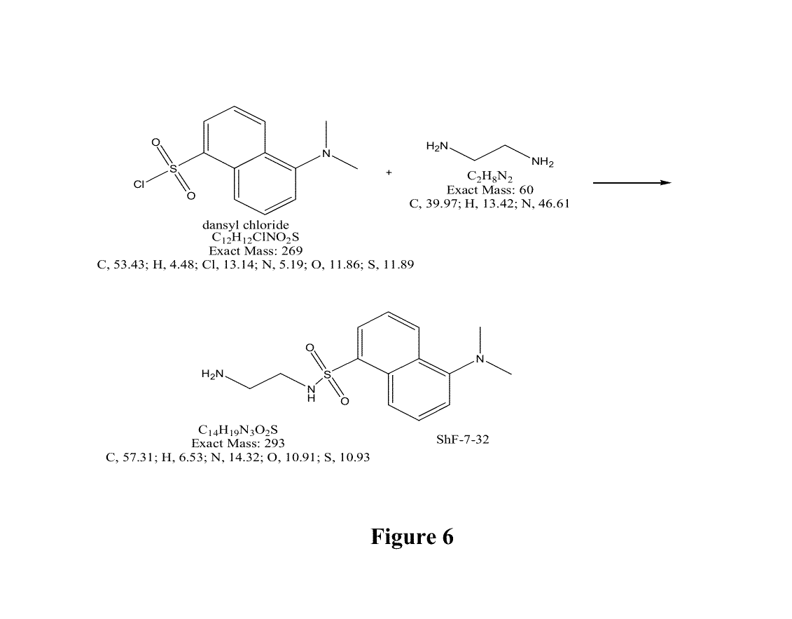

Dansyl ethylenediamine was prepared by mixing a solution of dansyl chloride (200 mg, 0.74 mmol) in dichloromethane (6 ml) with 1,2-ethylenediamine (6.5 ml, 445 mg, and 7.42 mmol) while stirring and cooling in ice. The mixture was stirred for 1 hr and acidified with dilute HCl and extracted with dichloromethane (2×20 ml). The aqueous layer was isolated and made basic (pH 9) using 10M NaOH and again extracted with DCM (2×20 ml). The organic layer was dried over Na₂SO₄, filtered and evaporated to dryness to form (2-aminoethyl)-dansylamide, ShF-7-32. See Scheme 2 (FIG. 6).

Drug particles complexation with oleylamine

The coating was prepared by two methods, in bulk and in solution.

1. Bulk antifungal agent: Olyelamine Complexation without solvent

5 g drug powder (0.015 mol active zinc) and 23 g of oleylamine-(technical 70%, 1:3.8 mol/mol) were refluxed at 95° C. for 3 days. After 3 days particles were filtrated using center flask, brownish particles were resulted. See scheme 3. ShF-7-55.

2. Bulk ANTIFUNGAL AGENT: Olyelamine complexation without solvent with 1% mol/mol Dansyl amine 5 g ANTIFUNGAL AGENT (0.015mol active zinc) and 23 g of oleylamine (1:3.8 mol/mol) and 46 mg of Dansyl ethylenediamine to give 1% mol/mol labelled were mixed at 95° C. for 2.5 days (60 hours). After 2.5 days particles were filtrated using center flask, were resulted and dried in active hood for 2 days, ShF-8-38A. This Material is repeating with fluorescent dye.

Drug powder Olyelamine Complexation in THF with 1% mol/mol Dansyl amine

5 g ANTIFUNGAL AGENT (0.015 mol active zinc) were dispersed in 50 ml THF and then 46 mg of Dansyl ethylenediamine to give 1% mol/mol labelled were refluxed at 95° C. for 1 hour and then 3 g of oleylamine (1:0.5 mol/mol) and continue mixing for 2.5 days (60 hours) at 95° C. After 2.5 days particles were filtrated using center flask, and washed with 50 ml THF and dried in active hood for 2 days, ShF-8-38B.

ANTIFUNGAL AGENT polymer coating stability in 15% SDS solution and tracing by FluoStar fluorimeter.

To a 15% SDS solution in DDW (0.5 ml, 20 mg of polymer coated ANTIFUNGAL AGENT particles) were added and left on an orbital shaker for 24 h at 30 rpm. After 24 h, samples were vigorously mixed for 1 min and left for the particles to precipitate and the upper liquid was filtrated through PTFE filters 0.2 μm. Aliquots of 200 μl of the clear solution were transferred to 96 wells plate and analyzed by fluorimeter (FluoStar) with excitation/emission at 390/590 nm and gain 140. Uncoated native particles of ANTIFUNGAL AGENT in 15% SDS were used as reference. Calibration curves were used to quantify the fluorescence in the SDS solution to determine stability of coating. The following samples were evaluated:

1—chitosan coating onto ANTIFUNGAL AGENT particles

2—crosslinked chitosan onto ANTIFUNGAL AGENT particles

3—crosslinked chitosan onto ANTIFUNGAL AGENT particles after imine bonds reduction

Dansyl labelled chitosan (in 1.2% Acetic acid in DDW),

For ANTIFUNGAL AGENT: Olyel amine complexes: Calibration curve of Dansyl ethylenediamine in 15% SDS were prepared from 0.01 mg-1.00 mg/ml. Using the prepared calibration curve coating stability of this complexes was studied in SDS.

Scanning Electron Microscopy (SEM)

Particles were placed on a conductive carbon paper and imaged using scanning electron microscopy (FEI E-SEM Quanta 2000) at an acceleration voltage of 30 KV. In parallel, energy-dispersive X-ray spectroscopy (EDx) analysis was applied for surface chemical characterization.

Smart Internal Reflection (iTR)

The polymer coated ANTIFUNGAL AGENT particles were analyzed by Smart iTR instrument, Nicolet iSlO (Thermo Scientific company, USA). Samples were placed directly on the diamond Nicolet and scanned in interval 500-4000 cm⁻¹, the spectra were evaluated by OMNIC software and calculation of similarity -spectra overlap.

Preparation of Mucoadhesive Particles Loaded with Steroids

Crosslinked Polymethacrylic acid (Carbopol 940) powder of 2-10 micron in size is swelled in a solution of 5% w/w dexamethasone in DMSO. The swelled powder was added to water to extract the DMSO. The wet powder is isolated and dried to produce powder loaded with dexamethasone. Alternatively, the Carbopol 940 is swelled in 5% aqueous solution of dexamethasone phosphate or triamcinolone succinate for 12 hours. The swelled powder was washed with ethanol and lyophilized to form microparticles loaded with steroids.

See Scheme 1 (FIG. 5) & Scheme 2 (FIG. 6).

9. Results:

FTIR and Energy-dispersive X-ray spectroscopy (EDx) were used to determine changes on ANTIFUNGAL AGENT surface. Native ANTIFUNGAL AGENT particles and coated ANTIFUNGAL AGENT particles were SEM visualized for their size and morphology. Similar particle size of 0.4-20 μm for both the coated and original ANTIFUNGAL AGENT particles was found.

Stability of coated ANTIFUNGAL AGENT particles in 15% SDS solution:

ANTIFUNGAL AGENT coating stability in 15% SDS (w/v) solution was determined using a calibration curve at a concentration interval of 0.1-12 mg/ml of fluorescent (dansyl) labelled polymer—calibration curve 1. ANTIFUNGAL AGENT coated particles exposed to SDS solution were analysed to determine the removal of fluorescent coating from coated ANTIFUNGAL AGENT particles. Calibration curves were performed for: Dansyl labelled chitosan (in 1.2% Acetic acid in DDW), and also calibration curve with interval 0.01-1.00 mg/ml in 15% SDS were prepared for Dansyl ethylenediamine for ANTIFUNGAL AGENT:Olyel amine complexes stability analysis

Using the above calibration curves the estimation of the stability of coated ANTIFUNGAL AGENT particles were performed.

Labled chitosan onto ANTIFUNGAL AGENT particles; crosslinked labled chitosan onto ANTIFUNGAL AGENT particles; crosslinked chitosan onto ANTIFUNGAL AGENT particles after imine bonds reduction; and Dansyl labelled chitosan (in 1.2% Acetic acid in DDW) was used.

The stability is satisfactory.

### REFERENCES

1. Senior, B. A. et al. Long-term results of functional endoscopic sinus surgery. The Laryngoscope 108, 151-157 (1998).

2. Young, J., Frenkiel, S., Tewfik, M. A. & Mouadeb, D. A. Long-term outcome analysis of endoscopic sinus surgery for chronic sinusitis. American journal of rhinology 21, 743-747, doi:10.2500/ajr.2007.21.3108 (2007).

3. Bhattacharyya, N. Clinical outcomes after revision endoscopic sinus surgery. Archives of otolaryngology--head & neck surgery 130, 975-978, doi:10.1001/archotol.130.8.975 (2004).

4. Ramadan, H. H., Sanclement, J. A. & Thomas, J. G. Chronic rhinosinusitis and biofilms. Otolaryngology--head and neck surgery : official journal of American Academy of Otolaryngology-Head and Neck Surgery 132, 414-417, doi:10.1016/j.otohns 2004.11.011 (2005).

5. Sanclement, J. A., Webster, P., Thomas, J. & Ramadan, H. H. Bacterial biofilms in surgical specimens of patients with chronic rhinosinusitis. The Laryngoscope 115, 578-582, doi:10.1097/01.mlg.0000161346.30752.18 (2005).

6. Bendouah, Z., Barbeau, J., Hamad, W. A. & Desrosiers, M. Biofilm formation by Staphylococcus aureus and Pseudomonas aeruginosa is associated with an unfavorable evolution after surgery for chronic sinusitis and nasal polyposis. Otolaryngology—-head and neck surgery : official journal of American Academy of Otolaryngology-Head and Neck Surgery 134, 991-996, doi:10.1016/j.otohns 2006.03.001 (2006).

7. Palmer, J. Bacterial biofilms in chronic rhinosinusitis. The Annals of otology, rhinology & laryngology. Supplement 196, 35-39 (2006).

8. Cohen, M. et al. Biofilms in chronic rhinosinusitis: a review. American journal of rhinology & allergy 23, 255-260, doi:10.2500/ajra.2009.23.3319 (2009).

9. Shikani A, Kourelis K. Topical Membrane Therapy for Chronic Rhinosinusitis. Source: Peculiar Aspects of Rhinosinusitis. Pages 91-112. ISBN 978-953-307-763-5. Edited by: Gian Luigi Marseglia and Davide Paolo Caimmi. Published November 2011 http ://www.intechopen. com/articles/show/title/topical-membrane-therapy-for-chronic-rhinosinusitis

10. Liang J, Lane A P. Topical Drug Delivery for Chronic Rhinosinusitis. Curr Otorhinolaryngol Rep. 2013 Mar. 1; 1(1):51-60. Epub 2012 Dec. 27.

11. Harvey R J, Goddard J C, Wise S K, Schlosser R J. Effects of endoscopic sinus surgery and delivery device on cadaver sinus irrigation. Otolaryngol Head Neck Surg. 2008; 139(1):137-42.

12. Olson D E, Rasgon B M, Hilsinger R L Jr. Radiographic comparison of three methods for nasal saline irrigation. Laryngoscope. 2002; 112(8 Pt 1):1394-8.

13. Snidvongs K, Chaowanapanja P, Aeumjaturapat S, Chusakul S, Praweswararat P. Does nasal irrigation enter paranasal sinuses in chronic rhinosinusitis? Am J Rhinol 2008; 22(5):483-6.

14. Grobler A, Weitzel E K, Buele A, et al. Pre- and postoperative sinus penetration of nasal irrigation. Laryngoscope. 2008; 118(11): 2078-81.

15. Wormald P J, Cain T, Oates L, Hawke L, Wong I. A comparative study of three methods of nasal irrigation. Laryngoscope. 2004; 114(12):2224-7.

16. Miller T R, Muntz H R, Gilbert M E, Orlandi R R. Comparison of topical medication delivery systems after sinus surgery. Laryngoscope. 2004; 114(2):201-4.

17. Valentine R, Athanasiadis T, Thwin M, Singhal D, Weitzel EK, Wormald P J. A prospective controlled trial of pulsed nasal nebulizer in maximally dissected cadavers. Am J Rhinol 2008; 22(4):390-4.

18. Merkus P, Ebbens F A, Muller B, Fokkens W J. The ‘best method’ of topical nasal drug delivery: comparison of seven techniques. Rhinology. 2006; 44(2):102-7.

19. Hyo N, Takano H, Hyo Y. Particle deposition efficiency of therapeutic aerosols in the human maxillary sinus. Rhinology. 1989; 27(1):17-26.

20. Laube BL. Devices for aerosol delivery to treat sinusitis. J Aerosol Med. 2007; 20(Suppl 1):S5-17. discussion S17-18.

21. Djupesland PG, Skretting A, Winderen M, Holand T. Breath actuated device improves delivery to target sites beyond the nasal valve. Laryngoscope. 2006; 116(3):466-72.

22. Hansen F S; Djupesland P G; Fokkens W J Preliminary efficacy of fluticasone delivered by a novel device in recalcitrant chronic rhinosinusitis Rhinology (2010), 48(3), 292-9

23. Moller W, Schuschnig U, Khadem Saba G, et al. Pulsating aerosols for drug delivery to the sinuses in healthy volunteers. Otolaryngol Head Neck Surg. 2010; 142(3):382-8.

24. Moller W, Schuschnig U, Meyer G, Mentzel H, Keller M. Ventilation and drug delivery to the paranasal sinuses: studies in a nasal cast using pulsating airflow. Rhinology. 2008; 46(3): 213-20.

25. Kern, R. C. et al. Perspectives on the etiology of chronic rhinosinusitis: an immune barrier hypothesis. American journal of rhinology 22, 549-559, doi:10.2500/ajr.2008.22.3228 (2008).

26. Zhang, Z. et al. Biofilms and mucosal healing in postsurgical patients with chronic rhinosinusitis. American journal of rhinology & allergy 23, 506-511, doi:10.2500/ajra.2009.23.3376 (2009).

27. Hekiert, A. M. et al. Biofilms correlate with TH1 inflammation in the sinonasal tissue of patients with chronic rhinosinusitis. Otolaryngology--head and neck surgery: official journal of American Academy of Otolaryngology-Head and Neck Surgery 141, 448-453, doi:10.1016/j.otohns 2009.06.090 (2009).

28. Kilty, S. J. & Desrosiers, M. Y. Are biofilms the answer in the pathophysiology and treatment of chronic rhinosinusitis? Immunology and allergy clinics of North America 29, 645-656, doi:10.1016/j.iac.2009.07.005 (2009).

29. Beule A G, Steinmeier E, Kaftan H, et al. Effects of a dexamethasone-releasing stent on osteoneogenesis in a rabbit model. Am J Rhinol Allergy. 2009; 23(4):433-6.

30. Bleier B S, Kofonow J M, Hashmi N, Chennupati S K, Cohen N A. Antibiotic eluting chitosan glycerophosphate implant in the setting of acute bacterial sinusitis: a rabbit model. Am J Rhinol Allergy. 2010; 24(2):129-32.

31. Huvenne W, Zhang N, Tijsma E, et al. Pilot study using doxycycline-releasing stents to ameliorate postoperative healing quality after sinus surgery. Wound Repair Regen. 2008; 16(6): 757-67.

32. Catalano P J, Thong M, Weiss R, Rimash T. The MicroFlow spacer: a drug-eluting stent for the ethmoid sinus. Indian J Otolaryngol Head Neck Surg. 2011; 63(3):279-84.

33. Murr A H, Smith T L, Hwang P H, et al. Safety and efficacy of a novel bioabsorbable, steroid-eluting sinus stent. Int Forum Allergy Rhinol 2011; 1(1):23-32.

34. Kennedy, D. The PROPEL® steroid-releasing bioabsorbable implant to improve outcomes of sinus surgery. Expert Review of Respiratory Medicine (2012), 6(5), 493-498.

35. Cario, Elke Nanotechnology-based drug delivery in mucosal immune diseases: hype or hope? Mucosal Immunology (2012), 5(1), 2-3Ensign, Laura M.; Schneider, Craig; Suk, Jung Soo; Cone, Richard; Hanes, Justin Mucus

36. Penetrating Nanoparticles: Biophysical Tool and Method of Drug and Gene Delivery. Advanced Materials (Weinheim, Germany) (2012), 24(28), 3887-3894.

Other embodiments will be apparent to those skilled in the art from consideration of the specification and practice of the embodiments disclosed herein. It is intended that the specification and examples be considered as exemplary only, with a true scope and spirit of the invention being indicated by the following claims.

Wwhat is claimed is:

1. A pharmaceutically acceptable composition, comprising one or more mucoadhesive polymers coating, dispersing, or dissolving one or more active ingredients.

2. The pharmaceutically acceptable composition of claim 1, wherein the one or more active ingredients are in the form of solid particles having an average size ranging from 0.050 to 15 microns (μm).

3. The pharmaceutically acceptable composition of claim 1, wherein the amount of one or more active ingredients ranges from 1 to 50 percent by weight (w/w %) of the one or more active ingredients and one or more mucoadhesive polymers.

4. The pharmaceutically acceptable composition of claim 1, wherein the one or more active ingredients are chosen from anti-inflammatory agents, antimicrobial active agents, antihistamines, and nasal decongestants.

5. The pharmaceutically acceptable composition of claim 1, wherein the one or more active ingredients are chosen from anti-inflammatory agents and antimicrobial/antifungal/antiinfective active agents.

6. The pharmaceutically acceptable composition of claim 1, wherein the one or more active ingredients is a bacteriophage.

7. The pharmaceutically acceptable composition of claim 1, wherein the one or more active ingredients is a combination of a bacteriophage and/ or an anti-inflammatory agent and/or antimicrobial, antifungal, and antiinfective active agent .

8. The pharmaceutically acceptable composition of claim 7, wherein the solid pharmaceutically acceptable carrier is coated with one or more second mucoadhesive polymers

9. The pharmaceutically acceptable composition of claim 1, wherein dispersion of the active ingredients occurs following a concentration gradient, with the highest concentration farthest away from the bio-film and the lowest concentration at the interface.

10. The pharmaceutically acceptable composition of claim 1, wherein the one or more mucoadhesive polymers are at least partially cross linked.

11. A method of treating rhinosinusitis, comprising contacting the nasal or sinus mucosa with an effective amount of a pharmaceutically acceptable composition, comprising one or more mucoadhesive polymers coating one or more active ingredients.

12. The method of claim 11, wherein the one or more active ingredients are chosen from anti-inflammatory agents, antimicrobial or antiinfective active agents, antihistamines, and nasal decongestants.

13. The method of claim 11, wherein the one or more active ingredients are chosen from anti-inflammatory agents and antimicrobial or antiinfective active agents.

14. The method of claim 13, wherein the anti-inflammatory agent is dispersed or dissolved in or the mucoadhesive polymer.

15. The method of claim 11, which is dispersed in a solid pharmaceutically acceptable carrier.

16. The method of claim 15, wherein the solid pharmaceutically acceptable carrier is coated with one or more second mucoadhesive polymers.

17. The method of claim 16, wherein the one or more second mucoadhesive polymers are at least partially cross linked.

18. The method of claim 11, wherein the one or more mucoadhesive polymers are at least partially cross linked.

19. A method of treating rhinosinusitis, comprising contacting the nasal or sinus mucosa with an effective amount of a pharmaceutically acceptable composition, comprising one or more mucoadhesive polymers coating one or more active ingredients, wherein the one or more active ingredients are chosen from antimicrobial or other antiinfective active agents and viral bacteriophages;
wherein an anti-inflammatory agent is dispersed or dissolved in or the mucoadhesive polymer.

20. The method of claim 19, wherein the antimicrobial active agents are chosen from antibiotics, antifungals, and anti-virals and the viral bacteriophages are chose from those belonging to a family chosen from ampullaviridae, bicaudaviridae, clavaviridae, corticoviridae, cystoviridae, fuselloviridae, globuloviridae, guttaviridae, inoviridae, leviviridae, microviridae, plasmaviridae, and tectiviridae. wherein an anti-inflammatory agent is chosen from steroid anti-inflammatories.
